(19) United States
(12) Patent Application Publication
(10) Pub. No.: US 2022/0023353 A1
(43) Pub. Date: Jan. 27, 2022

(54) COMPOSITION FOR IMPROVING MEMORY, OR COMPOSITION FOR PREVENTING AND TREATING ALZHEIMER'S DISEASE, COMPRISING ANGELICA ROOT EXTRACT AND SILKWORM PRODUCT HAVING SILK PROTEIN

(71) Applicants: REPUBLIC OF KOREA (RURAL DEVELOPMENT ADMINISTRATION), Jeollabuk-do (KR); QBM CO., LTD., Seoul (KR)

(72) Inventors: Sang Deok JI, Gyeonggi-do (KR); Kee Young KIM, Jeollabuk-do (KR); Nam Suk KIM, Jeollabuk-do (KR); You Young JO, Jeollabuk-do (KR); Su Bae KIM, Jeollabuk-do (KR); Sung Wan KIM, Incheon (KR); Jong Gil KIM, Jeollabuk-do (KR); Young Guk KIM, Gyeonggi-do (KR); Young Ho KO, Gyeonggi-do (KR); A Young KIM, Incheon (KR); Bo Hae CHOI, Gyeonggi-do (KR); Phuong NGUYEN, Gyeonggi-do (KR); Hyun Woo PARK, Gyeonggi-do (KR); Joo Won SUH, Gyeonggi-do (KR); Hui JIN, Seoul (KR); Mi Kyung SONG, Seoul (KR)

(21) Appl. No.: 17/294,567

(22) PCT Filed: Nov. 15, 2019
(86) PCT No.: PCT/KR2019/015688

§ 371 Date: May 17, 2021

(30) Foreign Application Priority Data
Nov. 15, 2018 (KR) 10-2018-0140505

(51) Int. Cl. A61K 35/64; A61K 36/232; A61K 38/17; A61P 25/28; A23L 33/105; A23L 33/17; A23C 9/152; A23L 2/02; A23L 2/66

(52) U.S. Cl. CPC A61K 35/64; A61K 36/232; A61K 38/1767; A61P 25/28; A23V 2002/00; A23L 33/17; A23C 9/1526; A23L 2/02; A23L 2/66; A23L 33/105

(57) ABSTRACT

The present invention relates to a composition for improving memory, or a composition for preventing and treating Alzheimer's disease, or a method for preparing said compositions, or a treatment method, wherein said compositions are characterized by comprising a cooked silkworm product and an Angelica root extract as effective components. As the present invention provides compositions using an Angelica root extract and a silkworm product rich in silk proteins and functional materials derived from mulberry leaves to improve memory and prevent or treat Alzheimer's disease, various health supplement products or drugs using the present invention are expected to be developed and commercialized, making it possible to fundamentally prevent and treat aging and dementia associated with aging, which are arising as a major problem in modern societies, and also to provide benefits of memory improvements in various other demographics, such as students, students studying for exams, office workers, etc.

### TECHNICAL FIELD

The present invention relates to a composition for improving memory or a composition for preventing or treating Alzheimer's dementia, in which the composition includes a steamed silkworm processed product having silk protein and an Angelica extract, and more particularly, to a composition for improving memory or composition for preventing or treating Alzheimer's dementia, in which the composition includes a steamed silkworm processed product having silk protein and an Angelica extract, the composition showing more significant efficacy than a single substance by combining two substances showing the effect of improving memory and preventing Alzheimer's dementia.

### BACKGROUND ART

In Korea, the annual fertility rate is rapidly decreasing. In 2017, the fertility rate was 1.05, reaching a record low, which is 0.53 less than the average of 1.68 in 2015 of the Organization for Economic Cooperation and Development. The number of newborns born in December 2017 was 25,000, less than the number of deaths of 26,900. In 2018, the number of newborns was further reduced, and the number of newborns from January to April decreased to 117,300, which is predicted to decrease to less than 1.0. This low fertility rate is predicted to advance the time when Korea's total population declines from 2031 to 2027. Accordingly, the proportion of the elderly population is expected to increase rapidly.

As of 2017, the number of older people aged 65 or older in Korea was 7 million, 14% of the total population. If the above-described fertility rate declines and the aging trend continues, the aging population is expected to increase rapidly by about 41.0% of the total population by 2060. The problem is that older people over 65 suffer from one or more chronic diseases for the rest of their lives. Among them, the disease that the elderly fear most is dementia, which has not yet been cured. Dementia is largely divided into Alzheimer's dementia, vascular dementia, and Parkinson's disease dementia, among which more than 60% suffer from Alzheimer's disease (AD). To date, no treatment or method has been developed to prevent or cure Alzheimer's dementia.

According to the analysis data of Korean National Institute of Dementia, the number of dementia patients in 2017 is predicted to reach 724,000 in 2017, 1,680,000 in 2040, and 2,127,000 in 2050. As a result, the cost of dementia management is expected to increase rapidly. The cost of dementia management is expected to increase rapidly in 2012, with KRW 10.3 trillion already being invested in management expenses, KRW 78.4 trillion in 2040, and KRW 133.6 trillion in 2050. Accordingly, according to the report on the results of the dementia prevalence survey conducted by Seoul National University Hospital in 2008, it is predicted that if the onset of dementia is delayed by two years, the prevalence will decrease by 20% after 40 years. Therefore, when converted as of 2040, it is predicted that the national dementia management costs of KRW 15 trillion will be reduced.

Meanwhile, the current treatment for dementia is a method aimed at alleviating symptoms rather than solving the underlying cause, and it is necessary to develop a substance for suppressing, delaying, or treating the onset.

Further, in Korea, as of 2015, the school-age population reached 7,559,000, so many people are devoting themselves to their studies. In addition to these students, university graduates and office workers can't go far from studying to prepare for various types of exams. In response to this, various kinds of products for enhancing memory have been released, but the effect is insignificant, and there are few products that perfectly respond to the needs of consumers.

### DISCLOSURE

### Technical Problem

The present invention is directed to providing a composition for improving memory or composition for preventing or treating Alzheimer's dementia, in which the composition includes a steamed silkworm processed product having silk protein and an Angelica extract having a more remarkably increased effect of preventing dementia or improving memory.

### Technical Solution

A pharmaceutical composition or food composition for improving memory and for preventing and improving Alzheimer's dementia of the present invention for achieving the above object is characterized by comprising a steamed silkworm processed product and Angelica extract.

The steamed silkworm processed product is characterized in that the whole silkworm having silk protein is heated for 100 minutes to 150 minutes with steam heat of 80° C. to 125° C., capable of being consumed for diet in a state in which silk protein is included.

The steamed silkworm processed product and the Angelica extract are characterized in that it is composed of a weight ratio of 1:0.1 to 1.

The technical problems to be achieved by the present invention are not limited to the technical problems mentioned above, and other technical problems that are not mentioned can be clearly understood by those of ordinary skill in the technical field to which the present invention belongs from the following description.

### Advantageous Effects

As the present invention provides compositions using an Angelica extract and a silkworm product rich in silk proteins and functional materials derived from mulberry leaves to improve memory and prevent or treat Alzheimer's disease, various health supplement products or drugs using the present invention are expected to be developed and commercialized, making it possible to fundamentally prevent and treat aging and dementia associated with aging, which is arising as a major problem in modern societies and also to provide benefits of memory improvements in various other demographics, such as students, students studying for exams, office workers, etc.

### DESCRIPTION OF DRAWINGS

FIG. 1 illustrates the life expectancy enhancement effect of a normal group Drosophila according to the treatment of a composition of the present invention comprising a steamed mature silkworm product and an Angelica gigas extract.

FIG. 2 illustrates the life expectancy enhancement effect of an Alzheimer's dementia (AD) model Drosophila according to the treatment of a composition of the present invention comprising a steamed mature silkworm product and an Angelica gigas extract.

FIG. 3 illustrates the neuron synapse structure enhancement effect according to the treatment of a composition of the present invention comprising a steamed mature silkworm product and an Angelica gigas extract.

FIG. 4 illustrates nerve cell protective activity according to the treatment of a composition of the present invention comprising a steamed mature silkworm product and an Angelica gigas extract.

FIG. 5 illustrates the activity enhancement effect of mitochondrial complexes 1 to 4 according to the treatment of a composition of the present invention comprising a steamed mature silkworm product and an Angelica gigas extract.

FIG. 6 illustrates the enhancement effect of ATP production ability according to the treatment of a composition of the present invention comprising a steamed mature silkworm product and an Angelica gigas extract.

FIG. 7 illustrates the memory enhancement effect of the short-term memory loss agent-treated mice according to the treatment of a composition of the present invention comprising a steamed mature silkworm product and an Angelica gigas extract.

FIG. 8 illustrates a comparison of the spatial memory enhancement effect according to the treatment of a composition of the present invention comprising a steamed mature silkworm product and an Angelica gigas extract, in the Y-maze test.

Data represent mean±S.E.M (N=6).

(#: p<0.05 Control vs Con+Sco

*: p<0.05 Con+Sco vs SW+Sco or CDG+Sco or COM+Sco or RG+Sco or DO+Sco)

(SW: steamed mature silkworm product powder, CDG: Angelica gigas Nakai, COM: a combination of steamed mature silkworm product and Angelica gigas Nakai, RG: memory-improving red ginseng product, DO: donepezil, SCO: scopolamine)

FIG. 9 illustrates a comparison of the spatial memory enhancement effect according to the treatment of a combination of steamed mature silkworm product and Angelica gigas extract of the composition of the present invention in the Morris water maze test.

Data represent mean±S.E.M (N=5).

(##: p<0.01 Control vs Con+Sco

*: p<0.05 Con+Sco vs SW+Sco or CDG+Sco or COM+Sco or RG+Sco or DO+Sco

**: p<0.01 Con+Sco vs SW+Sco or CDG+Sco or COM+Sco or RG+Sco or DO+Sco

***: p<0.001 Con+Sco vs SW+Sco or CDG+Sco or COM+Sco or RG+Sco or DO+Sco)

(SW: steamed mature silkworm product powder, CDG: Angelica gigas, COM: a combination of steamed mature silkworm product and Angelica gigas Nakai, RG: memory-improving red ginseng product, DO: donepezil, SCO: scopolamine)

FIG. 10 illustrates the self-control ability enhancement effect of 3×TG dementia mice according to the treatment of a composition of the present invention comprising a steamed mature silkworm product and an Angelica gigas extract.

FIG. 11 illustrates the recognition ability enhancement effect for a new object of 3×TG dementia mice according to the treatment of a composition of the present invention comprising a steamed mature silkworm product and an Angelica gigas extract.

FIG. 12 illustrates the sociality enhancement effect of 3×TG dementia mice according to the treatment of a composition of the present invention comprising a steamed mature silkworm product and an Angelica gigas extract.

### MODES OF THE INVENTION

Hereinafter, the present invention is described in detail, and detailed descriptions of known functions and configurations that may unnecessarily obscure the subject matter of the present invention are omitted.

According to an aspect of the present invention, the present invention is a composition or food composition for improving memory; and a composition or a food composition for preventing or treating Alzheimer's dementia, the composition comprising a steamed silkworm processed product including silk proteins and functional materials derived from mulberry leaves and Angelica extract.

That is, the present invention is characterized in that it relates to the use of improving memory or preventing and treating Alzheimer's dementia of a steamed silkworm processed product containing a silk protein and a functional substance derived from mulberry leaves and an Angelica extract.

In other words, the steamed silkworm processed product containing the silk protein means that all silkworms having silk proteins such as trimolter silkworm and tetramolter silkworm are prepared through the heating process, and is used interchangeably with the steamed mature silkworm product or HONG JAM in the present specification.

That is, the silkworm having silk protein may be preferably selected from 5th day of 3rd instar silkworm to mature silkworm stage of tetramolter silkworm. Here, as 5th days of 3rd instar silkworm, post-5th day of 4th instar silkworm can be preferably used, and mature silkworm can be more preferably used. This is because, although 5th day of 3rd instar silkworm can be used, the silkworm has less silk protein and more silkworm dung than the post-5th day of 4th instar silkworm. Meanwhile, the term silkworm of the mature silkworm stage refers to a silkworm that is full of silk protein in the body of the silkworm and before the cocoon is built. It is most suitable for use as an active ingredient in pharmaceutical compositions or food compositions (health functional foods) because most of the silkworm dung is discharged. In addition, as the silkworm variety of the steamed silkworm processed product, any variety of silkworm may be used, but among them, any one of white silk variety, yellow silk variety (Golden silk), light green silk variety, and light red silk variety is used. More preferably, the yellow silk variety exhibits an excellent recovery effect, so that it is best to apply the yellow silk variety thereto.

In the present invention, in order to enable dietary intake of all silk proteins contained in silkworms as described above, the silkworms are processed and manufactured in the form of processed products and used as an active ingredient in a pharmaceutical composition or food composition (including health functional foods). At this time, the silkworm is heated by steam heat and is preferably heated for 100 minutes to 150 minutes by steam heat at 80° C. to 125° C. so that all silk protein is contained. In other words, if it is prepared for more than 150 minutes with steam heat below 80° C., the preparation time is too long, and there is a possibility that the active ingredients of the silkworm itself containing silk protein may be destroyed. If it is prepared for less than 100 minutes at greater than 125° C., the temperature is too high, which may adversely affect the active ingredients of the silkworm itself.

After the steamed process, it can be dried by a conventional method, and it can be prepared and used in powder form, and it is preferable to dry and powder it through anyone selected from hot air drying or vacuum freeze-drying. In order to minimize the loss of active ingredients, the vacuum freeze drying method is most suitable.

The pulverization may be performed using any pulverizer such as a home blender, hammer mill, roller mill, freeze pulverizer, etc., but a roller mill or freeze pulverization method is preferable for minimizing sample loss, preventing component distortion, and fine powdering. It is very difficult to pulverize the powder with a size of less than 0.1 mm when pulverized with a general home blender or hammer mill. However, if it is repeatedly pulverized with a roller mill, it is possible to finely powder the size of the powdered particles up to an average of about 10 μm. Thus, it is expected to be beneficial to the intake and the product.

In addition, after the preparation as a form of the powder, the steamed silkworm processed product powder may be prepared in the form of an extract, and at this time, it may be prepared by extraction with water, a lower alcohol having 1 to 4 carbon atoms, or a mixed solvent thereof. The lower alcohol having 1 to 4 carbon atoms may be selected from the group consisting of methanol, ethanol, propanol, isopropanol, butanol, and iso-butanol. The water, a lower alcohol having 1 to 4 carbon atoms, or a mixed solution thereof may be used in a volume of 1 to 40 times (1 to 40 liters per 1 kg), preferably 5 to 40 times, based on the weight of the steamed silkworm processed product powder. The extraction conditions of the extract may be 1 hour to 48 hours at 20° C. to 100° C., and the process may be repeated up to 1 to 4 times.

In another method, the steamed silkworm processed product powder is extracted and concentrated with water, a lower alcohol having 1 to 4 carbon atoms, or a mixed solvent thereof, and then suspended by adding water, to the extract, preferably 1 to 1000 times, more preferably 1 to 500 times, and most preferably 1 to 50 times based on the weight of the extract, a solvent selected from the group consisting of hexane, chloroform, ethyl acetate, and butanol may be added to the suspension to prepare the fraction.

The preparation temperature of the extract or the fraction thereof may be 20 to 100° C. but is not limited thereto. The extraction or fractionation time is not particularly limited but is preferably extracted within 10 minutes to 2 days, and a conventional extraction device, an ultrasonic grinding extractor, or a fractionator may be used as an extraction device. The thus prepared extract or fraction may be dried with hot air, dried under reduced pressure, or freeze-dried to remove the solvent. In addition, the extract or fraction may be purified and used using column chromatography. The chromatography includes one selected from silica gel column chromatography, LH-20 column chromatography, ion exchange resin chromatography, medium pressure liquid chromatography, thin layer chromatography (TLC), silica gel vacuum liquid chromatography, and high-performance liquid chromatography.

In addition, Angelica gigas Nakai, one of the active ingredients of the present invention, is a perennial herbaceous plant belonging to the genus Angelica Linnaeus of Apiaceae. It has been reported to have an anemia recovery effect and immunity enhancing effect. Regarding the blood circulation, blood vessel contraction inhibitory effect and platelet aggregation inhibition, angiogenesis promotion, antioxidant function, blood alcohol concentration reduction, antioxidant and antithrombotic function, analgesic function, liver function protection, anticancer function, central inhibitory function, blood vessel relaxation effect, and whitening effect studies have been reported. Angelica gigas Nakai, Angelica acutiloba Kitagawa, or Angelica sinensis Diels may be used as the Angelica, but preferably, it is best to use Angelica gigas Nakai.

This Angelica is characterized in that it is prepared and used in the form of an extract in order to obtain the desired effect of the present invention, and the extract can be isolated according to a conventional method known in the art.

When the Angelica extract used in the present invention is obtained by treating Angelica with an extraction solvent, various extraction solvents may be used. Preferably, a polar solvent or a non-polar solvent can be used. Suitable polar solvents include (i) water, (ii) alcohols (preferably methanol, ethanol, propanol, butanol, normal-propanol, iso-propanol, normal-butanol, 1-pentanol, 2-butoxyethanol. or ethylene glycol), (iii) acetic acid, (iv) dimethyl-formamide (DMFO), and (v) dimethyl sulfoxide (DMSO). Suitable non-polar solvents include acetone, acetonitrile, ethyl acetate, methyl acetate, fluoroalkane, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethylamine, ether, carbon tetrachloride, and THF.

More preferably, the extraction solvent used in the present invention includes (a) water, (b) anhydrous or hydrous lower alcohol having 1-4 carbon atoms (methanol, ethanol, propanol, butanol, etc.), (c) mixed solvent with the lower alcohol and water, (d) acetone, (e) ethyl acetate, (0 chloroform, (g) butyl acetate, (h) 1,3-butylene glycol, (i) hexane and (j) diethyl ether. Most preferably, the extract of the present invention is obtained by treating Angelica with water, methanol, ethanol, or a combination thereof.

The term “extract” used herein has the meaning commonly used as a crude extract in the art as described above, but broadly includes a fraction obtained by additionally fractionating the extract. In other words, the Angelica extract includes not only those obtained by using the above-described extraction solvent but also those obtained by additionally applying a purification process thereto. For example, the Angelica extract of the present invention also includes fractions obtained through additionally performed various purification methods such as fractions obtained by passing the extract through an ultrafiltration membrane having a certain molecular weight cut-off value and fractions obtained by separation of various chromatography (made for separation according to size, charge, hydrophobicity or affinity).

The Angelica extract used in the present invention may be prepared in a powder state by additional processes such as distillation under reduced pressure, freeze-drying and spray drying.

In one embodiment of the present invention, a lower alcohol such as methanol or ethanol, preferably 95% ethanol, is added to the root diameter of Angelica, and then they are extracted at 5° C. to 80° C., preferably 30° C. to 55° C. for 15 minutes to 48 hours, preferably 30 minutes to 12 hours and subsequently dried under reduced pressure.

The present invention provides a composition in which the steamed silkworm processed product and the Angelica extract are mixed. Thus, compared to the application of each of the single substances, the mixed composition increases the life expectancy and health of both the normal control animal model and the Alzheimer's dementia model, prevents memory loss, improves synapse of neuron structure, protects nerve cells, enhances mitochondrial activity to increase the production of ATP, improves spatial memory, improves postural control ability, enhances the ability to perceive new objects, and improves sociality. Therefore, it has been confirmed that it shows a more significant effect of preventing Alzheimer's dementia and improving memory. In the present invention, as an active ingredient of a pharmaceutical composition or a food composition (health functional food) for improving memory or for preventing and treating Alzheimer's dementia, the two substances are used together. More preferably, the steamed silkworm processed product and the Angelica extract are mixed in a ratio of 1:0.1 to 1 by weight. When the Angelica extract is mixed in less than 0.1 weight ratio based on the weight of the steamed silkworm processed product, it is difficult to achieve the significant effect aimed at the present invention, and when it exceeds 1 weight ratio, the increased efficacy according to the excess content may not be obtained.

In the present specification, the terms “a mixture of the steamed mature silkworm product powder+the Angelica gigas Nakai extract” and “combination” used while referring to “a composition in which the steamed mature silkworm processed product and the Angelica extract are mixed together” mean the active ingredient of the present invention, and they are used interchangeably.

Further, the mixture of steamed silkworm processed product and Angelica extract may be added to the pharmaceutical composition of the present invention in an amount of 0.001% by weight to 100% by weight. The pharmaceutical compositions may be formulated and used in the form of oral dosage forms such as powders, granules, tablets, capsules, suspensions, emulsions, syrups and aerosols, external preparations, suppositories, and sterile injectable solutions according to a conventional method. Carriers, excipients, and diluents may be included in the pharmaceutical composition and include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils. The formulation is prepared using diluents or excipients such as fillers, extenders, binders, wetting agents, disintegrants, and surfactants that are usually used. Solid preparations for oral administration include tablets, pills, powders, granules, capsules, and the like, and these solid preparations are prepared by mixing at least one excipient, such as starch, calcium carbonate, sucrose, lactose, gelatin, or the like. In addition to simple excipients, lubricants such as magnesium stearate and talc are also used. Liquid preparations for oral administration include suspensions, liquid solutions, emulsions, syrups, etc. In addition to water and liquid paraffin, which are commonly used as simple diluents, various excipients such as wetting agents, sweeteners, fragrances, and preservatives may be included. Formulations for parenteral administration include sterilized aqueous solutions, non-aqueous solutions, suspensions, emulsions, lyophilized formulations, and suppositories. As the non-aqueous solvent and suspension, propylene glycol, polyethylene glycol, vegetable oil such as olive oil, and injectable ester such as ethyl oleate may be used. As a base for suppositories, witepsol, macrogol, tween 61, cacao butter, laurin paper, glycerogelatin, and the like may be used.

The dosage of the pharmaceutical composition of the present invention varies depending on the age, gender, and weight of the individual to be treated, the specific disease or pathological state to be treated, the severity of the disease or pathological state, the route of administration, and the judgment of the prescriber. Dosage determination based on these factors is within the level of one of skill in the art, and dosages generally range from 100 mg/kg/day to 5,000 mg/kg/day. A more preferred dosage is 1,000 mg/kg/day to 3,000 mg/kg/day. Administration may be once a day or may be divided several times. The dosage does not limit the scope of the present invention in any way.

The pharmaceutical composition of the present invention may be administered to mammals such as mice, livestock, and humans by various routes. All methods of administration may be expected, and the composition may be administered, for example, by oral, rectal, intravenous, intramuscular, subcutaneous, intrauterine dural or cerebrovascular injection. The mixture of the present invention has almost no toxicity and side effects so that it is a drug that may be safely used even when taken for a long period for prophylactic purposes.

In this case, the mixture consisting of the steamed silkworm processed product and Angelica extract is preferably included in an amount of 1% by weight to 100% by weight, more preferably 10% by weight to 90% by weight based on the total weight of the composition in terms of the weight of the powder as an effective content for showing the desired efficacy of the present invention.

Further, the present invention may provide a food composition for improving memory or for preventing and improving Alzheimer's dementia, including a mixture consisting of the steamed silkworm processed product and Angelica extract. At this time, the mixture consisting of the steamed silkworm processed product and Angelica extract may be used in an amount of 1% by weight to 100% by weight in the food composition of the present invention. The food composition of the present invention is formed in for example tablets, capsules, pills or liquids, and is included in various drinks, meats, sausages, bread, candies, snacks, noodles, ice cream, dairy products, soups, ionic beverages, beverages, alcoholic beverages, gum, tea or vitamin complexes.

Further, according to another aspect of the present invention, the present invention provides a method for improving memory, the method comprising administering to a subject a composition comprising the above-described steamed silkworm processed product and Angelica extract.

Further, according to still another aspect of the present invention, the present invention provides a method for preventing or treating Alzheimer's dementia, the method comprising administering to a subject a composition comprising the above-described steamed silkworm processed product and Angelica extract.

Further, according to further another aspect of the present invention, the present invention provides a method of preparing a composition for improving memory, the method comprising mixing the above-described steamed silkworm processed product and Angelica extract.

Further, according to yet another aspect of the present invention, the present invention provides a method for preparing a composition for preventing or treating Alzheimer's dementia, the method comprising mixing the above-described steamed silkworm processed product and Angelica extract.

Since the methods of the present invention use the above-described composition, overlapped contents are excluded in order to avoid excessive complexity of the present specification.

As described above, the present invention is expected to be of great help in improving memory by enhancing the brain function of all citizens, including the elderly and youth, including students at each level of school suffering from academic stress. In addition, the incidence of neurological diseases including Alzheimer's dementia is effectively reduced to be expected to contribute greatly to the improvement of the life quality of people living in the aging era, and furthermore, to be expected to contribute greatly to the reduction of national dementia management costs.

Hereinafter, the present invention is described in more detail with reference to Examples, but these Examples are only for illustrative purposes and are not intended to limit the protective scope of the present invention.

### Example 1. Preparation of Mixture Consisting of a Steamed Matured-Silkworm Processed Product and an Angelica Extract

1) Preparation of Steamed Matured-Silkworm Processed Product (Steamed Mature Silkworm Product; HONG JAM)

Goldensilk silkworm variety having yellow-color silk protein (a hybrid produced by crossing the Japanese race bred Jam311 and the Chinese race bred Jam312) was bred. About 7th day of 5th instar, the matured Golden silk silkworm was heated for 120 minutes to 130 minutes with steam at 100° C. to obtain a steamed matured-silkworm processed product (a steamed mature silkworm product or a boiled mature silkworm product) from Goldensilk silkworm variety.

The steamed mature-silkworm processed product from Golden silk silkworm was freeze-dried with vacuum for 24 hours to prepare a powder form.

2) Preparation of Ethanol Extract of Angelica

Korean Angelica gigas Nakai was used. After weighing the raw materials, 70% (v/v) alcohol of 10 times the amount of the original was added thereto. The extraction was performed twice at 85° C. for 4 hours each. Then, the extract was filtered and concentrated, and 40% (w/w, based on total weight) dextrin was mixed. A powder form was prepared through spray drying.

3) Preparation of Mixture

A mixture was prepared by mixing 0.2 g/kg of the steamed silkworm processed product powder of 1) and 0.16 g/kg of the Angelica ethanol extract of 2) and applied as a sample of the following experiments.

### Experimental Example 1. Comparison of Component Analysis of Non-Steamed Mature Silkworm and Steamed Silkworm Processed Product or the Steamed Silkworm Processed Products by Variety

1) Comparison of Component Analysis of Non-Steamed Mature Silkworm and Steamed Silkworm Processed Product

The main components were analyzed through a hammer mill pulverized substance having a particle size of 0.1 mm of Non-steamed mature silkworm and Steamed silkworm processed product, respectively. The results are shown in Tables 1 and 2 below.

TABLE 1

| | | Steamed mature | Non-steamed mature |
|---|---|---|---|
| | | silkworm product | silkworm |
| Classification | | (white silk) | (white silk) |
| Component | Moisture | 2.85 | 4.15 |
| (g/100 g) | Crude protein | 68.86 | 62.43 |
| | Crude fat | 10.99 | 11.66 |
| | Crude ash | 3.26 | 3.63 |
| Amino acid | Cysteine | 0.427 | 0.463 |
| (%) | (CYS) | | |
| | Methionine | 0.644 | 0.756 |
| | (MET) | | |
| | Serine | 6.757 | 6.194 |
| | (SER) | | |
| | Glutamic acid | 3.824 | 4.071 |
| | (GLU) | | |
| | Glycine | 12.335 | 9.265 |
| | (GLY) | | |
| | Alanine | 9.778 | 7.460 |
| | (ALA) | | |
| | Leucine | 1.983 | 2.114 |
| | (Leu) | | |
| | Lysine | 2.193 | 2.396 |
| | (Lys) | | |
| | Histidine | 1.083 | 1.160 |
| | (His) | | |
| | Arginine | 1.958 | 2.123 |
| | (Arg) | | |
| | Proline | 1.148 | 1.217 |
| | (Pro) | | |
| | Tryptophan | 0.425 | 0.437 |
| | (Trp) | | |

TABLE 2

| | | Steamed mature | Non-steamed mature |
|---|---|---|---|
| | | silkworm product | silkworm |
| Classification | | (white silk) | (white silk) |
| Fatty acid | Oleic acid | 31.22 | 31.13 |
| (%) | (C18:lΩ9) | | |
| | Eicosenoic acid | 0.23 | 0.28 |
| | (C20:lΩ9) | | |
| | Unsaturated fatty | 66.79 | 67.49 |
| | acids | | |
| | - Single fatty | 32.31 | 32.23 |
| | acids | | |
| Inorganic | Ca(Calcium) | 198.90 | 197.83 |
| ingredients | Fe(Iron) | 3.17 | 8.49 |
| (mg/100 g) | K(Potassium) | 1045.44 | 1176.37 |
| | Mg(Magnesium) | 199.25 | 211.94 |
| | Mn(Manganese) | 1.60 | 1.70 |
| | P(Phosphorus) | 688.27 | 749.79 |

Through the above results, it was confirmed that the contents of various components in the mature silkworm were changed variously as the steamed silkworm processed product by heating. It can be seen that there will be a change in the pharmacological effect of the steamed silkworm processed product due to the change in the components of the steamed silkworm processed product.

2) Comparison of Component Analysis Through Roller Mill Grinding Method for Each Silkworm Variety

The components of the steamed silkworm processed product by silkworm varieties were analyzed and compared through the roller mill grinding method, which is a grinding method with little loss of raw materials and little change in composition.

The results are shown in Tables 3 to 8 below.

TABLE 3
Content of general components according to silkworm varieties
of Steamed mature silkworm product (Unit: g/100 g)

| Classification | Baegokjam | Golden silk | Younokjam | Red silk variety |
|---|---|---|---|---|
| Moisture | 4.29 ± 0.03c | 2.31 ± 0.05b | 2.34 ± 0.02b | 1.71 ± 0.08a |
| Crude protein | 71.92 ± 0.34bc | 69.36± 0.23a | 73.13 ± 0.5c | 70.89 ± 0.055b |
| Crude fat | 13.57 ± 0.11a | 15.59 ± 1.005a | 14.05 ± 0.065a | 15.21 ± 0.925a |
| Crude ash | 3.48 ± 0.235a | 3.43 ± 0.06a | 3.50 ± 0.13a | 3.97 ± 0.16b |
| Crude fiber | 2.96 ± 0.05ab | 3.10 ± 0.03ab | 2.78 ± 0.03a | 3.50 ± 0.01b |

Sample were analyzed by one-way ANOVA followed by DUNKAN test
a.b.c Different letters indicate significant differences at p < 0.05

TABLE 4
Content of amino acids according to silkworm varieties
of Steamed mature silkworm product(Unit: g/100 g)

| Classification | Baegokjam | golden silk | Younokjam | Red silk variety |
|---|---|---|---|---|
| Cysteine (CYS) | 0.424 ± 0.002^{a} | 0.442 ± 0^{b} | 0.433 ± 0.001^{ab} | 0.437 ± 0.005^{b} |
| Methionine (MET) | 0.690 ± 0.005a | 0.778 ± 0.006c | 0.725 ± 0.004b | 0.730 ± 0.006b |
| Aspartic acid(ASP) | 5.129 ± 0.035b | 5.092 ± 0.046b | 5.299 ± 0.059c | 4.871 ± 0.02a |
| Threonine (THR) | 2.489 ± 0.015b | 2.492 ± 0.019b | 2.503 ± 0.03b | 2.407 ± 0.005a |
| Serine (SER) | 7.707 ± 0.029b | 6.946 ± 0.106a | 7.548 ± 0.059b | 7.024 ± 0.046a |
| Glutamic acid (GLU) | 4.759 ± 0.05ab | 4.858 ± 0.043b | 4.869 ± 0.067b | 4.641 ± 0.009a |
| Glycine (GLY) | 12.810 ± 0.017b | 11.353 ± 0.238a | 12.438 ± 0.044b | 11.842 ± 0.091a |
| Alanine (ALA) | 10.220 ± 0.021b | 9.342 ± 0.181a | 10.177 ± 0.025b | 9.687 ± 0.068a |
| Valine (VAL) | 2.248 ± 0.017a | 2.345 ± 0.018b | 2.275 ± 0.006a | 2.233 ± 0.002a |
| Isoleucine (Ile) | 1.064 ± 0.007a | 1.145 ± 0.006b | 1.082 ± 0.013a | 1.080 ± 0.006a |
| Leucine (Leu) | 1.814 ± 0.02a | 1.992 ± 0.018c | 1.888 ± 0.016b | 1.816 ± 0.007a |
| Tyrosine (Tyr) | 4.830 ± 0.013b | 4.563 ± 0.074a | 4.781 ± 0.015b | 4.443 ± 0.067a |
| Phenylalanine(Phe) | 1.942 ± 0.029a | 2.061 ± 0.017b | 1.977 ± 0.024ab | 1.937 ± 0.016a |
| Lysine (Lys) | 2.544 ± 0.01a | 2.669 ± 0.037b | 2.621 ± 0.016ab | 2.536 ± 0.013a |
| Histidine (His) | 1.151 ± 0.005a | 1.182 ± 0.012a | 1.138 ± 0.012a | 1.235 ± 0.012b |
| Arginine (Arg) | 2.118 ± 0.006a | 2.171 ± 0.035a | 2.134 ± 0.006a | 2.108 ± 0.005a |
| Proline (Pro) | 1.606 ± 0.014a | 1.884 ± 0.043b | 1.788 ± 0.008b | 1.805 ± 0.018b |
| Tryptophan (Trp) | 0.506 ± 0.008a | 0.532 ± 0.005ab | 0.542 ± 0.007b | 0.517 ± 0.001ab |

Sample were analyzed by one-way ANOVA followed by DUNKAN test
a.b.c Different letters indicate significant differences at p < 0.05

TABLE 5
Content of fatty acids according to silkworm varieties
of Steamed mature silkworm product (Unit: %)

| Classification | Baegokjam | Golden silk | Younokjam | Red silk variety |
|---|---|---|---|---|
| Myristic acid(C14:0) | 0.15 ± 0.01a | 0.14 ± 0.01a | 0.15 ± 0.00a | 0.20 ± 0.01b |
| Palmitic acid(C16:0) | 21.87 ± 0.77a | 24.24 ± 0.53a | 22.03 ± 1.24a | 24.94 ± 1.24a |
| Palmitoleic | 0.74 ± 0.09a | 0.83 ± 0.01a | 0.73 ± 0.01a | 0.73 ± 0.01a |
| acid(C16:ln7) | | | | |
| Stearic acid(C18:0) | 7.38 ± 0.22a | 8.05 ± 0.05ab | 7.73 ± 0.36a | 8.60 ± 0.36b |
| Oleic acid(C18:ln9) | 25.95 ± 1.82a | 25.60 ± 0.38a | 25.39 ± 2.37a | 26.19 ± 2.37a |
| Linoleic acid(C18:2n6) | 8.16 ± 0.16bc | 7.65 ± 0.52b | 9.03 ± 0.30c | 5.66 ± 0.30a |
| γ-Linoleic | 0.11 ± 0.0ab | 0.16 ± 0.02bc | 0.08 ± 0.01a | 0.17 ± 0.01c |
| acid(C18:3n6) | | | | |
| Linolenic acid(C18:3n3) | 35.55 ± 0.91a | 33.29 ± 1.85a | 34.77 ± 0.21a | 33.45 ± 0.21a |
| Eicosenoic | 0.11 ± 0.01b | 0.06 ± 0.01a | 0.10 ± 0.00b | 0.06 ± 0.00a |
| acid(C20:ln9) | | | | |
| Unsaturated fatty acids | 70.62 | 67.59 | 70.10 | 66.26 |
| Saturated fatty acids | 29.38 | 32.41 | 29.90 | 33.74 |

Sample were analyzed by one-way ANOVA followed by DUNKAN test
a.b.c Different letters indicate significant differences at p < 0.05

TABLE 6
Content of inorganic components according to silkworm
varieties of Steamed mature silkworm product

| Classification | Baegokjam | Golden silk | Younokjam | Red silk variety |
|---|---|---|---|---|
| Ca(mg/100 g) | 190.01 ± 6.85b | 168.75 ± 1.90a | 180.69 ± 1.13ab | 203.73 ± 7.76c |
| Cu(mg/100 g) | 0.59 ± 0.03b | 0.49 ± 0.01a | 0.48 ± 0.01a | 0.50 ± 0.02a |
| Fe(mg/100 g) | 2.64 ± 0.09b | 1.94 ± 0.03a | 1.97 ± 0.04a | 3.91 ± 0.48c |
| K(mg/100 g) | 608.90 ± 17.48a | 586.00 ± 51.24a | 650.12 ± 14.75a | 647.93 ± 13.19a |
| Mg(mg/100 g) | 183.34 ± 4.02ab | 189.22 ± 2.84b | 172.69 ± 2.09a | 176.90 ± 3.53a |
| Mn(mg/100 g) | 1.36 ± 0.01a | 1.35 ± 0.01a | 1.22 ± 0.01b | 1.35 ± 0.03a |
| Na(mg/100 g) | 34.83 ± 2.11a | 39.54 ± 1.69a | 35.75 ± 1.49a | 39.50 ± 1.65a |
| P(mg/100 g) | 388.73 ± 5.86a | 405.47 ± 6.89a | 388.68 ± 3.28a | 386.61 ± 8.47a |
| Zn(mg/100 g) | 6.36 ± 0.18c | 5.05 ± 0.09a | 5.64 ± 0.06b | 5.32 ± 0.16ab |
| S(mg/kg) | 4171.63 ± 18.55a | 4652.50 ± 21.79b | 4318.63 ± 18.54c | 4557.00 ± 69.90d |

Sample were analyzed by one-way ANOVA followed by DUNKAN test
a.b.c Different letters indicate significant differences at p < 0.05

TABLE 7
Content of vitamins according to silkworm varieties of Steamed mature silkworm product

| Classification | Baegokjam | Golden silk | Younokjam | Red silk variety |
|---|---|---|---|---|
| Beta-carotene (ug/100 g) | 1107.24 ± 69.48a | 4026.07 ± 100.52b | 1188.98 ± 19.95a | 7304.22 ± 157.50c |
| Vitamin B1 (mg/100 g) | 0.57 ± 0.01ab | 0.59 ± 0.01b | 0.54 ± 0.00a | 0.63 ± 0.02c |
| Vitamin B2(mg/100 g) | 5.95 ± 0.15b | 4.63 ± 0.09a | 5.36 ± 0.05a | 6.04 ± 0.1c |
| Vitamin B3(mg/100 g) | 4.15 ± 0.05a | 4.94 ± 0.12b | 4.06 ± 0.05a | 4.73 ± 0.16b |
| Vitamin B6(mg/100 g) | 0.55 ± 0.01a | 0.52 ± 0.01a | 0.51 ± 0.01a | 0.67 ± 0.01b |
| Folic acid(mg/100 g) | 0.46 ± 0.01a | 0.72 ± 0.01d | 0.52 ± 0.01b | 0.59 ± 0.01c |
| Vitamin C(mg/100 g) | 10.19 ± 0.11b | 11.63 ± 0.14c | 9.26 ± 0.11a | 12.33 ± 0.05d |

Sample were analyzed by one-way ANOVA followed by DUNKAN test
a.b.c Different letters indicate significant differences at p < 0.05

TABLE 8
Content of functional components according to silkworm
varieties of Steamed mature silkworm product

| Classification | Baegokjam | Golden silk | Younokjam | Red silk variety |
|---|---|---|---|---|
| Total polyphenols | 773.43 ± 11.91ab | 744.45 ± 15.06a | 881.01 ± 18.09c | 822.10 ± 26.83b |
| (mg/100 g) | | | | |
| Total flavonoids | 452.60 ± 13.79ab | 423.59 ± 3.63a | 552.64 ± 21.69c | 494.46 ± 16.07b |
| (mg/100 g) | | | | |
| GABA | 2.28 ± 0.06b | 1.96 ± 0.008a | 2.51 ± 0.10c | 4.59 ± 0.06b |
| (mg/100 g) | | | | |

Sample were analyzed by one-way ANOVA followed by DUNKAN test
a.b.c Different letters indicate significant differences at p < 0.05

As a result of the above experiment, there is a difference in the components contained in the steamed mature silkworm powder by silkworm variety, and based on this result, it is predicted that the following effects are different for each variety. In other words, as a result of analyzing the components of the steamed silkworm processed product by silkworm variety, the steamed silkworm processed product contains a large amount of crude protein, amino acids such as glycine, alanine, and serine, unsaturated fatty acids such as linolenic acid, and vitamins such as beta-carotene. Trans fat, heavy metals such as mercury, and sugars such as sucrose, which are harmful to the human body, were not detected. In addition, compared to 3rd day of 5th instar dried silkworms and un-steamed mature silkworm products, the steamed mature silkworm product had a very high content of crude protein and amino acids such as glycine, alanine, and serine. By silkworm varieties, all the same ingredients were detected in all varieties, such as Baegokjam, Golden silk, Younokjam, and red silk varieties. In the content comparison by component, it is expected that there will be a difference in the functionality of each silkworm variety because it is contained differently for each variety.

### Experimental Example 2. Confirmation of the Improvement Effect of the Life Expectancy of Normal Group Drosophila by Treatment with the Mixture of Example 1 of the Present Invention

### 1. Experimental Method

It is reported that life expectancy decreases significantly when suffering from Alzheimer's dementia. Drosophila model for the normal control group not suffering from dementia and the experimental group suffering from Alzheimer's dementia was used to conduct the test on the steamed mature silkworm product powder, the Angelica gigas Nakai extract, and a mixture of the steamed mature silkworm product powder and the Angelica gigas Nakai extract (hereinafter referred to as “combination”) of Example 1.

First, a study was conducted to confirm the synergistic effect of increasing the life expectancy of the combination of steamed mature silkworm product powder and Angelica gigas Nakai extract (Example 1) for the normal control group not suffering from Alzheimer's dementia (control group).

### 2. Experimental Result

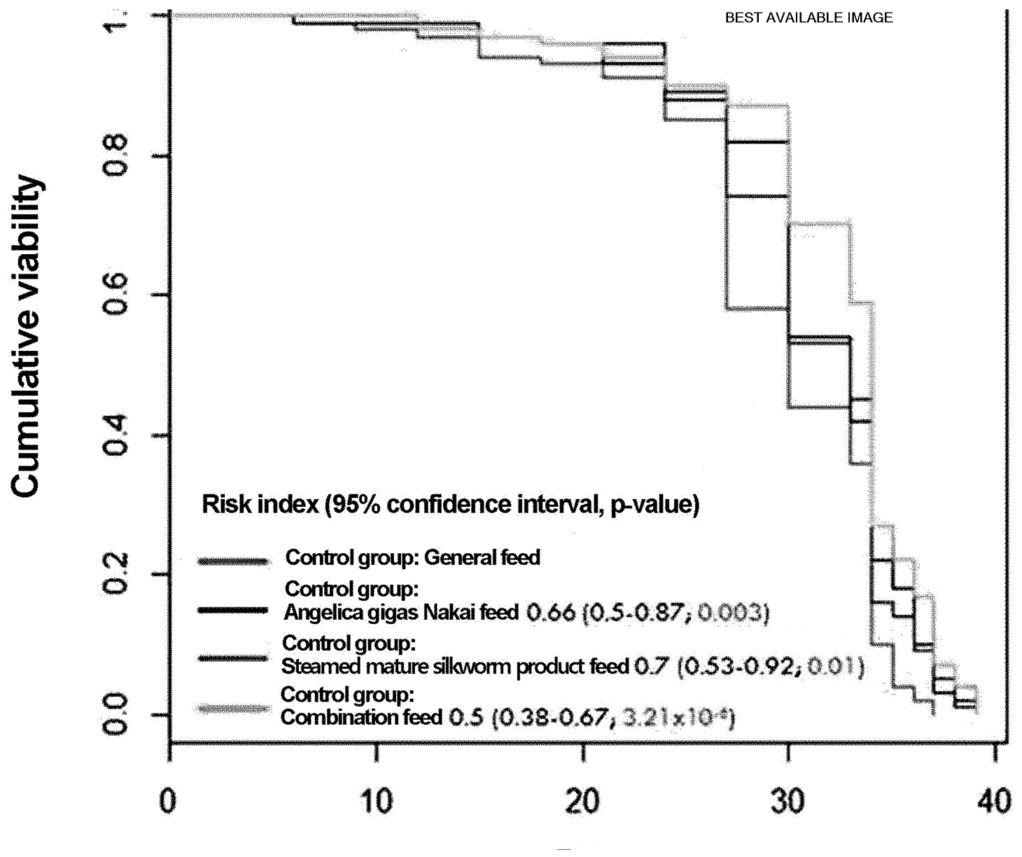

The experimental results are shown in FIG. 1 and Table 9 below.

TABLE 9

| | Average Life | |
|---|---|---|
| Treatment group | (days) | Ratio |
| General feed | 29.29 | — |
| Angelica gigas Nakai extract | 31.18 | 6.8% increase |
| Steamed mature silkworm product powder | 31.02 | 5.9% increase |
| Combination of Steamed mature silkworm | 32.32 | 10.3% increase |
| product and Angelica gigas Nakai | | |
| (Example 1) | | |

As shown in Table 9 and FIG. 1, it was confirmed that life expectancy of the combination of steamed mature silkworm product powder/Angelica gigas nakai extract (Example 1) was significantly increased to be 1.5 to 1.7 times (3.5 to 4.4% p) the increase in life expectancy of the experimental group treated with only the steamed mature silkworm product powder or Angelica gigas Nakai extract individually.

Specifically, the life expectancy of the normal Drosophila that consumed only general feed was 29.29 days, but the life expectancy of the experimental group that consumed the Angelica gigas Nakai extract was increased by 6.8% to 31.18 days. The risk index was 0.66 (95% Confidence interval (CI): 0.5 to 0.87, p value=0.003), which was statistically significantly decreased. In the case of the experimental group that ingested the steamed mature silkworm powder, the life expectancy was 31.02 days, which was 5.9% higher than that of the general feed, and the risk index was 0.7 (95% CI: 0.53 to 0.92, p-value=0.01), which was statistically significantly reduced compared to the general feed. On the other hand, in the case of animals ingesting feed containing a combination of steamed mature silkworm product powder and Angelica gigas Nakai extract (Example 1), the life expectancy was increased by 10.3% to 32.32 days, and the risk index was reduced to be 0.5 (95% CI=0.38 to 0.67, p. value=3.21×10⁻⁶). These results show that the life expectancy of the experimental group treated with the combination (Example 1) was increased due to a mutual synergistic effect which is 1.5 to 1.7 times (3.5 to 4.4% p) compared to the life expectancy increase rate of the group treated with the steamed mature silkworm product powder or Angelica gigas Nakai extract individually, showing a significant increase in life expectancy and a further decrease in risk index.

### Experimental Example 3. Confirmation of the Improvement Effect of the Life Expectancy of the Alzheimer's Dementia (AD) Model Drosophila by Treatment with the Mixture of Example 1 of the Present Invention

### 1. Experimental Method

Alzheimer's dementia is a disease that causes behavioral changes due to loss of intellectual abilities such as memory and thinking and is known to account for more than 60% of all cases of dementia. Alzheimer's dementia is reported to reduce life expectancy either directly or indirectly. Therefore, a study was conducted using an Alzheimer's dementia model Drosophila that expresses three genes that cause Alzheimer's dementia in humans.

In other words, a Drosophila model that simultaneously expresses Amyloid precursor protein (APP), beta site amyloid precursor protein cleaving enzyme 1 (BACE1), and Microtubule-associated protein tau (MAPT), which are the causative genes of human Alzheimer's disease, was cultured using general feed with 10% combination of steamed mature silkworm product powder and Angelica gigas Nakai extract (Example 1). After the cultured 100 or more adult Drosophila were divided into one vial by 10, the number of living Drosophila was checked at intervals of 3 days until all Drosophila died. Drosophila was reared under conditions of 28° C. and 60% relative humidity. For statistical analysis, Kaplan-Meyer survival analysis was performed to investigate statistical differences.

Thus, the effect of increasing the life expectancy of the test group suffering from Alzheimer's disease (AD) was tested. At this time, the effect of increasing the life expectancy of the Drosophila was confirmed in the same experimental method as in Experimental Example 2, except that the effect of increasing the life expectancy of the Alzheimer's dementia model Drosophila was confirmed instead of the normal group Drosophila.

### 2. Experimental Result

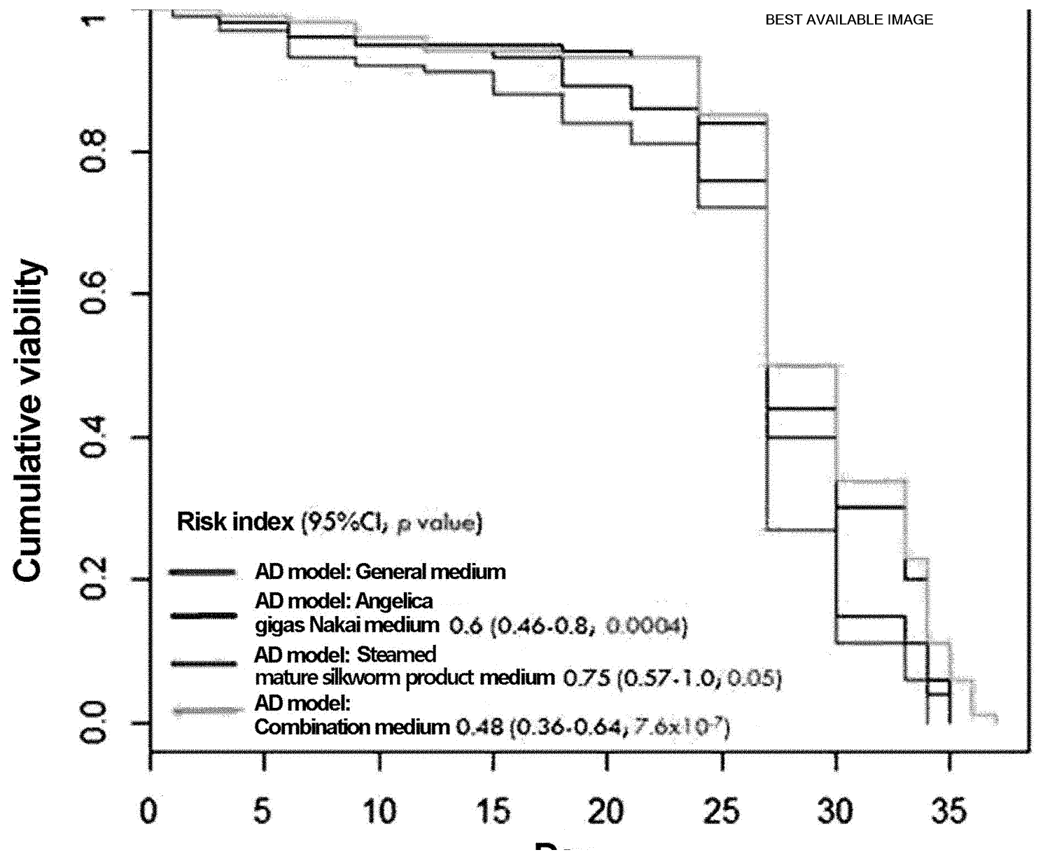

The experimental results are shown in FIG. 2 and Table 10 below.

TABLE 10

| | Average Life | |
|---|---|---|
| Treatment group | (days) | Ratio |
| General feed | 25.22 | — |
| Angelica gigas Nakai extract | 26.01 | 3.1% increase |
| Steamed mature silkworm product powder | 26.58 | 5.4% increase |
| Combination of Steamed mature silkworm | 28.49 | 13.0% increase |
| product and Angelica gigas Nakai | | |
| (Example 1) | | |

As shown in Table 10 and FIG. 2, the life expectancy of the Alzheimer's dementia model Drosophila was decreased by 13.9% to 25.22 days compared to the normal control group (29.29 days). It was confirmed that the reduced life expectancy of the Alzheimer's dementia model Drosophila was increased by treatment with the Angelica gigas Nakai extract alone, the steamed mature silkworm product powder alone, or the combination of steamed mature silkworm product powder and Angelic gigas Nakai extract (Example 1).

In particular, the combination of steamed mature silkworm product powder and Angelica gigas Nakai extract (Example 1) was increased 2.4 to 4.2 times compared to administration as a single substance, which is a surprising synergistic effect close to that of the normal group.

Specifically, the life expectancy of the Alzheimer's dementia model Drosophila, which fed with only general feed, was 25.22 days, while the life expectancy of the Alzheimer's dementia model Drosophila, fed with a feed supplemented with Angelica gigas Nakai extract, was increased by 3.1% to 26.01 days, and the risk index was 0.6 (95% CI=0.46 to 0.8, p-value=0.0004), which is statistically significantly decreased. Further, the life expectancy of the Alzheimer's dementia model Drosophila, fed with a feed supplemented with the steamed mature silkworm product powder, was increased by 5.4% to 26.58 days, and the risk index was 0.57 (95% CI=0.57 to 1.0, p-value=0.05), which is statistically significantly decreased.

Compared to this, the life expectancy of the Alzheimer's dementia model Drosophila, fed with a feed supplemented with a combination of the steamed mature silkworm product powder and Angelica gigas Nakai was 28.49 days, which is an increase of 13.0% compared to that of the Alzheimer's dementia model Drosophila, fed with only general feed. The risk index was also 0.48 (95% CI=0.36 to 0.64, p-value=7.6×10⁻⁷), showing a statistically significant decrease.

Summarizing the results, the Alzheimer's dementia model Drosophila fed with a combination of the steamed mature silkworm product powder and Angelica gigas Nakai had significant effects. The life expectancy was increased by 2.4 to 4.2 times (7.6 to 9.9% p) compared to the increase in life expectancy of Drosophila fed with only Angelica gigas Nakai extract (increased 3.1%) or the steamed mature silkworm product powder (increased 5.4%). This result shows that the life expectancy of the Alzheimer's dementia model Drosophila administered with a combination of the steamed mature silkworm product powder and Angelica gigas Nakai (Example 1) was close to that of the normal group. It has been shown that the synergistic interaction between steamed mature silkworm product powder and Angelica gigas Nakai extract leads to significantly increased life expectancy and reduced risk index compared to feed added with each substance individually.

### Experimental Example 4. Confirmation of the Structural Change of the Neuron Synapse According to the Treatment with the Mixture of Example 1 of the Present Invention

### 1. Experimental Method

Alzheimer's dementia is known to be caused by an abnormal neuron synapse structure in the human brain. Therefore, a study was conducted on the changes in the neuron synapse structure of the normal control Drosophila and the Alzheimer's dementia model Drosophila.

First, it was tested for the changes in the neuron synapse structure of the normal control Drosophila model not suffering from Alzheimer's dementia. At this time, the mixture of Example 1 was added to the normal feed by adding 10% of the total amount of yeast, which is the main protein source, of the components of the normal feed to culture the normal control Drosophila. 3rd instar mature larva were dissected in a Ca²⁺-free dissection solution in the larval stage, which is the pre-adult stage. Then, it was fixed using a 0.1M phosphate buffer solution with an acidity of 7.2 containing 4% paraformaldehyde. The fixed samples were washed 3 times at 10 minute intervals using 0.01M phosphate buffered saline containing 0.1% Triton X-100. Then, neuron synapses were stained using anti-HRP-FITC. The number of neuron synapses was confirmed using a fluorescence microscope.

Further, it was tested for the changes in the neuron synapse structure of the Drosophila model who has Alzheimer's dementia using the above-mentioned experimental method.

### 2. Experimental Result

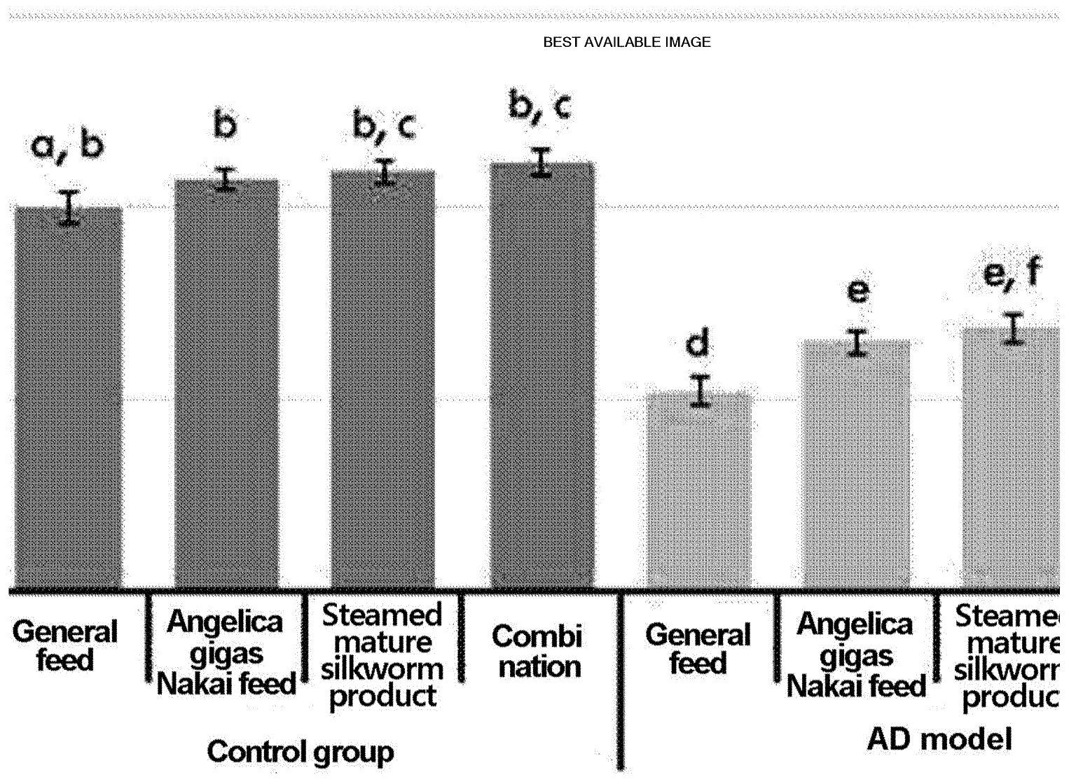

As a result of the above experiment, as shown in FIG. 3, when converting the neuron synapse structure of the normal control Drosophila in which only the general feed was fed to 100%, the experimental group in which only the general feed containing the Angelica gigas Nakai extract was consumed had a 7.2% increase in the number of neuron synapse, and the experimental group in which only the general feed containing the steamed mature silkworm product powder was fed a 9.3% increase in the number of neuron synapse.

Compared to this, in the case of Drosophila fed with a combination of the steamed mature silkworm product powder and Angelica gigas Nakai extract added to the general feed (Example 1), the number of neuron synapses was increased by 11.7%. Thus, a combination of the steamed mature silkworm product powder and Angelica gigas Nakai extract (Example 1) had a remarkable synergistic effect showing an increase rate of 1.3 to 1.6 times (2.4 to 4.5% p) compared to the increase rate of the neuron synapse of the experimental groups with which only Angelica gigas Nakai extract or only steamed mature silkworm product powder was fed.

Next, the result of testing the change in the neuron synapse structure of the Drosophila model who has Alzheimer's dementia confirmed that the Drosophila in the AD model, which was fed with only general feed, had 47.9% smaller number of neuron synapse compared to the normal control group.

Accordingly, it can be seen that the AD Drosophila model fed with only feed supplemented with Angelica gigas Nakai extract had 12.7% increased number of the neuron synapse compared to Drosophila fed with only general feed. It can be seen that the AD Drosophila model fed with feed supplemented with the steamed mature silkworm product powder had 16.5% increased number of the neuron synapse. Compared to these, the experimental group in which the AD Drosophila model was fed with feed including a combination of the steamed mature silkworm product powder and Angelica gigas Nakai extract (Example 1) had 22.1% increased number of the neuron synapse compared to the experimental group fed with only general feed. This shows a significant synergistic increase in neuron synapse, which was 1.3 to 1.7 times (5.6 to 9.4% p) higher than that of the experimental group fed with Angelica gigas Nakai extract alone or the steamed mature silkworm product powder alone.

These results show that the case where a combination of the steamed mature silkworm product powder and Angelica gigas Nakai extract (Example 1) was treated had a synergistic effect by interaction in both the normal control group and the Alzheimer's dementia model compared to the case where Angelica gigas Nakai extract alone or the steamed mature silkworm product powder alone was treated.

### Experimental Example 5. Confirmation of Neuronal Protective Activity by Treatment with the Mixture of Example 1 of the Present Invention

In order to test the neuronal protective effect according to the treatment of the mixture of Example 1 of the present invention, each neuronal protective activity of the steamed mature silkworm product alone (SW), Angelica gigas Nakai extract alone (AG), and a combination of the steamed mature silkworm product and Angelica gigas Nakai extract (SWA-1) was confirmed in the HT22 cell damage model induced by L-glutamate.

Cell viability was measured by 3-(4,5-dimethythiazol2-yl)-2,5-di-phenytetrazolium bromide (MTT, Sigma, USA) assay. The formazan is produced by converting MTT, a yellow water-soluble substrate, into deep blue water-insoluble formazan using the ability of living cells' mitochondria dehydrogenase. The amount of formazan is proportional to the number of living cells.

HT22 cells were incubated at constant temperature in a 96-well plate for 24 hours under a condition of 37° C. and 5% CO₂, and then treated with SW, AG and SWA-1, respectively at each concentration, and then the cells incubated for 24 hours were washed with PBS, and then MTT solution (5 mg/ml) was added and reacted for 4 hours. The produced formazan was dissolved in DMSO and measured at 570 nm.

### 2. Experimental Result

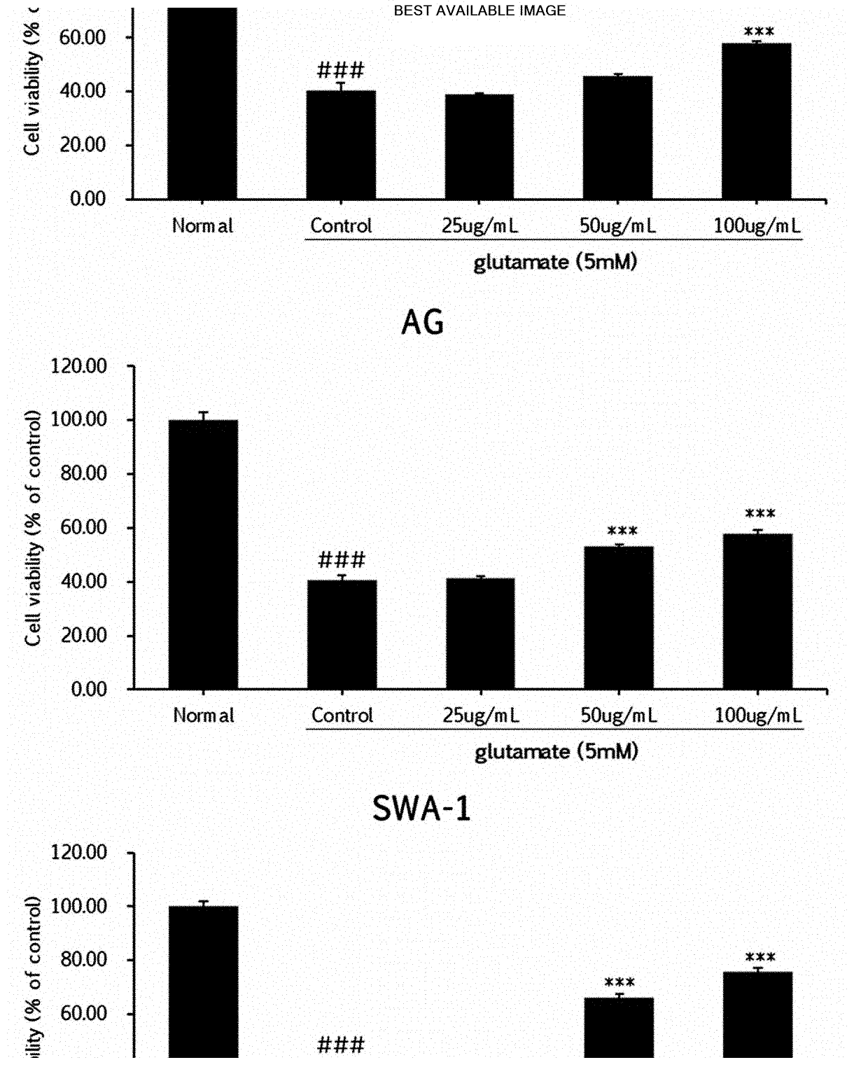

As a result of the above experiment, as shown in FIG. 4, compared to the control group in the case in which 50 ug/mL and 100 ug/mL respectively of steamed mature silkworm product alone (SW) were treated, the cell viability was increased by 5.33% and 17.49%, respectively compared to the control group. In the case in which 50 ug/mL and 100 ug/mL respectively of Angelica gigas Nakai extract alone (AG) were treated, the cell viability was increased by 12.55% and 17.23%, respectively. In the case in which 50 ug/mL and 100 ug/mL respectively of a combination of the steamed mature silkworm product and Angelica gigas Nakai extract (SWA-1) were treated, the cell viability was increased by 25.99% and 35.84%, respectively.

These results show that compared to the case where the Angelica gigas Nakai extract alone or the steamed mature silkworm product powder alone, the case treated with a combination of the steamed mature silkworm product powder and Angelica gigas Nakai extract (Example 1) had a synergistic effect due to the interaction in the neuronal damage model.

### Experimental Example 6. Confirmation of the Synergistic Effect of Activity of Mitochondrial Complexes 1 to 4 by the Treatment of the Mixture of Example 1 of the Present Invention

### 1. Experimental Method

It is known that although aging proceeds normally, the activity of mitochondria decreases so that the ability to produce ATP decreases. More importantly, mitochondrial dysfunction, which is the molecular cellular etiology commonly observed in Alzheimer's dementia patients, occurs earlier than in normal individuals.

Therefore, the change in the activity of the mitochondrial complexes 1 to 4 were compared for cases in which the normal control Drosophila and the Alzheimer's dementia model Drosophila, respectively, were raised and fed with general feed alone, general feed supplemented with Angelica gigas Nakai extract, general feed supplemented with the steamed mature silkworm product powder or general feed supplemented with a combination of the steamed mature silkworm product powder and Angelica gigas Nakai extract.

Specifically, functions from mitochondrial complexes I to IV were confirmed using normal Drosophila and Alzheimer's dementia Drosophila, fed with the mixture of Example 1. The main function of mitochondria is to produce ATP, which is the main energy material of cells, and ATP is produced by five respiratory complexes present in the inner membrane of mitochondria. Complexes I to IV generate a potential difference in the mitochondrial inner membrane by oxidative phosphorylation, and Complex V actually produces ATP. Therefore, a study was conducted to compare the activities of mitochondrial complexes 1 to 4.

For the assay of mitochondrial complex I activity, 10 ml of mitochondria extracted from the brain of adult Drosophila were added to 182 ml of activity assay buffer (25 mM PB, 0.35% BSA, 0.06 mM DCIP, 0.07 mM decylubiquinone, 1 mM AA). The resultant was mixed with 8 ml of 5 mM NADH, and reacted at 37° C. for 30 minutes. The change in absorbance was measured at 600 nm for 4 minutes at 30 second intervals, and the activity was measured in 5 repeated samples. For the assay of mitochondrial complex II activity, 184 ml of activity assay buffer (80 mM PB pH 7.8, 0.1% BSA, 2 mM EDTA, 0.2 mM ATP, 80 mM DCIP, 50 mM decylubiquinone, 1 mM AA, 3 mM rotenone) was added with 1 ml malonate and 10 ml of mitochondrial extract and reacted at 37° C. for 30 minutes. The change in absorbance was measured at 600 nm for 4 minutes at 30 second intervals, and the activity was measured in 5 repeated samples. For the assay of mitochondrial complex III activity, 80 ml of an activity assay buffer (50 mM Tris-HCl pH 7.5, 4 mM NaN₃, 0.1 mM decylubiquinone, 80 mM Succinate, 2 mM KCN, 3 mM rotenone, 40 mM Cytochrome C) was added with 10 ml of the mitochondrial extract and 10 ml of the enzyme reaction solution. The measurements were performed at 25° C. at 550 nm for 3 minutes at 30 second intervals. For the assay of mitochondrial complex IV activity, 188 ml of activity assay buffer (10 mM Tris-HCl pH 7.0, 120 mM KCl) was added with 10 ml Ferrocytochrome C substrate (0.22 mM Ferrocytochrome and 0.5 mM DTT), 2 ml of the mitochondrial extract and 2 ml of the enzyme reaction solution. The measurements were performed at 25° C. at 550 nm for 1 minute at 10 second intervals. The activity was measured in 5 repeated samples. The difference between groups was confirmed by ANOVA, and Tukey-Kramer post hoc analysis was performed.

### 2. Experimental Result

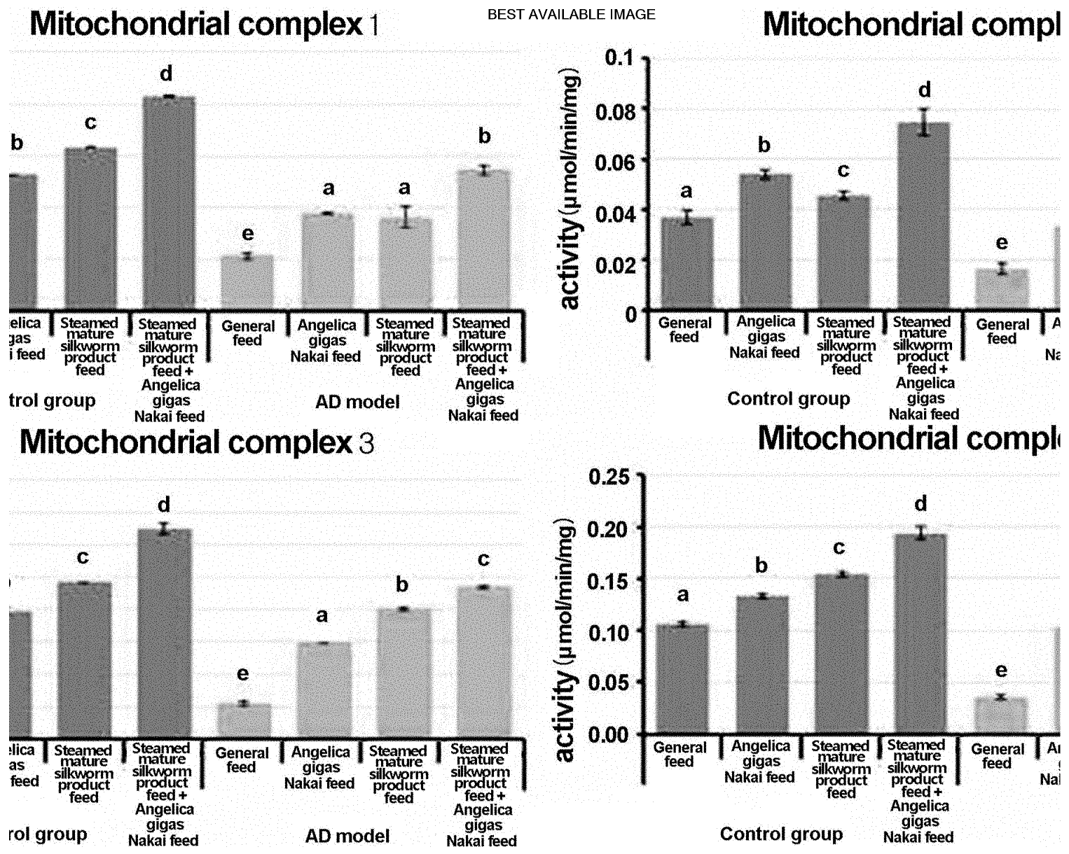

As shown in FIG. 5, the result of the above experiment confirmed that cases in which Drosophila was fed with a general feed supplemented with Angelica gigas Nakai extract alone, the steamed mature silkworm product powder alone, or a combination of the steamed mature silkworm product powder and Angelica gigas Nakai extract (Example 1) had an increase in the activities of mitochondrial complexes 1 to 4 in all of the Drosophila models of Alzheimer's dementia. In particular, the Drosophila model fed with a general feed supplemented with a combination of the steamed mature silkworm product powder and Angelica gigas Nakai extract (Example 1) had a significant increase in the activity.

1) First, the Activity of Mitochondrial Complex in the Normal Control Group not Suffering from Alzheimer's Dementia is Described in Detail as Follows.

For the activity of mitochondrial complex 1 of the normal control group, compared to the experimental group with which only general feed was fed, Drosophila of the experimental group fed with a feed supplemented with Angelica gigas Nakai extract was increased by 20.5%, and Drosophila of the experimental group fed with a feed supplemented with the steamed mature silkworm product powder was increased by 33.7%. On the other hand, Drosophila of the experimental group fed with a combination of the steamed mature silkworm product powder and Angelica gigas Nakai extract (Example 1) was increased by 45.5%. The result shows that a combination of the steamed mature silkworm product powder and Angelica gigas Nakai extract has a significantly high activity, which is 1.4 to 2.2 times (11.8 to 25.0% p), which is greater than the activity increase rate of mitochondrial complex 1 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of complex 1.

For the activity of mitochondrial complex 2 of the normal control group, compared to the experimental group with which only general feed was fed, Drosophila of the experimental group fed with a feed supplemented with Angelica gigas Nakai extract was increased by 45.9%, and Drosophila of the experimental group fed with a feed supplemented with the steamed mature silkworm product powder was increased by 23.1%. On the other hand, Drosophila of the experimental group fed with a combination of the steamed mature silkworm product powder and Angelica gigas Nakai extract (Example 1) was increased by 100.9%. The result shows that a combination of the steamed mature silkworm product powder and Angelica gigas Nakai extract has a significantly higher activity increase rate, which is 2.2 to 4.4 times (77.8 to 55.0% p), which is greater than the activity increase rate of mitochondrial complex 2 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 2.

Further, for the activity of mitochondrial complex 3 of the normal control group, compared to the experimental group with which only general feed was fed, Drosophila of the experimental group fed with a feed supplemented with Angelica gigas Nakai extract was increased by 29.0%, and Drosophila of the experimental group fed with a feed supplemented with the steamed mature silkworm product powder was increased by 57.7%. On the other hand, Drosophila of the experimental group fed with a combination of the steamed mature silkworm product powder and Angelica gigas Nakai extract was increased by 111.2%. The result shows that a combination of the steamed mature silkworm product powder and Angelica gigas Nakai extract (Example 1) has a significantly higher activity increase rate, which is 1.9 to 3.8 times (53.5 to 82.2% p), which is greater than the activity increase rate of mitochondrial complex 3 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 3.

Further, for the activity of mitochondrial complex 4 of the normal control group, compared to the experimental group with which only general feed was fed, Drosophila of the experimental group fed with a feed supplemented with Angelica gigas Nakai extract was increased by 24.1%, and Drosophila of the experimental group fed with a feed supplemented with the steamed mature silkworm product powder was increased by 43.7%. On the other hand, Drosophila of the experimental group fed with a combination of the steamed mature silkworm product powder and Angelica gigas Nakai extract (Example 1) was increased by 81.3%. The result shows that a combination of the steamed mature silkworm product powder and Angelica gigas Nakai extract has a significantly higher activity increase rate, which is 1.9 to 3.4 times (37.6 to 57.2% p), which is greater than the activity increase rate of mitochondrial complex 4 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 4.

Further, as a summary of mitochondrial complex activity in normal control Drosophila, the normal Drosophila fed with the steamed mature silkworm product powder alone or Angelica gigas Nakai extract alone had a great increase in the activity of mitochondrial complexes 1 to 4. On the other hand, the normal Drosophila fed with a combination of the steamed mature silkworm product powder and Angelica gigas Nakai extract (Example 1) had a significant increase in the activity of mitochondrial complexes 1 to 4 compared to the experimental group treated with single feed. Thus, it is determined that it can greatly contribute to the prevention of Alzheimer's dementia in the process of aging.

2) Next, after the Alzheimer's Dementia Model Drosophila was Fed with alone or in a combination of Angelica gigas as Nakai extract and the steamed mature silkworm product powder, the activities of mitochondrial complexes 1 to 4 were compared (FIG. 5).

The result confirmed that the activity of mitochondrial complexes 1 to 4 of the Alzheimer's dementia model Drosophila cultivated in feed with alone or in a combination of Angelica gigas Nakai extract and steamed mature silkworm product powder was significantly increased and that a combination shows a significantly increased rate compared to a single feed.

Specifically, for the activity of mitochondrial complex 1 of the Alzheimer's dementia model Drosophila, compared to the experimental group with which only general feed was fed, the experimental group fed with a feed supplemented with Angelica gigas Nakai extract was increased by 76.8%, and the experimental group fed with a feed supplemented with the steamed mature silkworm product powder was increased by 70.2%. On the other hand, the experimental group fed with a combination of the steamed mature silkworm product powder and Angelica gigas Nakai extract (Example 1) was increased by 155.3%. The result shows that a combination of the steamed mature silkworm product powder and Angelica gigas Nakai extract has a significantly high activity increase rate, which is 2.0 to 2.2 times (78.5 to 85.1% p), which is greater than the activity increase rate of mitochondrial complex 1 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 1.

For the activity of mitochondrial complex 2 of the Alzheimer's dementia model Drosophila, compared to the experimental group with which only general feed was fed, the experimental group fed with a feed supplemented with Angelica gigas Nakai extract was increased by 100.0%, and the experimental group fed with a feed supplemented with the steamed mature silkworm product powder was increased by 106.9%. On the other hand, Drosophila of the experimental group fed with a combination of the steamed mature silkworm product powder and Angelica gigas Nakai extract (Example 1) was increased by 214.0%. The result shows that a combination of the steamed mature silkworm product powder and Angelica gigas Nakai extract has a significantly higher activity increase rate, which is 2.0 to 2.1 times (107.1 to 114.0% p), which is greater than the activity increase rate of mitochondrial complex 2 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 2.

Further, for the activity of mitochondrial complex 3 of the Alzheimer's dementia model Drosophila, compared to the experimental group with which only general feed was fed, the experimental group fed with a feed supplemented with Angelica gigas Nakai extract was increased by 168.1%, and the experimental group fed with a feed supplemented with the steamed mature silkworm product powder was increased by 263.0%. On the other hand, the experimental group fed with a combination of the steamed mature silkworm product powder and Angelica gigas Nakai extract (Example 1) was increased by 324.1%. The result shows that a combination of the steamed mature silkworm product powder and Angelica gigas Nakai extract has a significantly higher activity increase rate, which is 1.2 to 1.9 times (61.1 to 156.0% p), which is greater than the activity increase rate of mitochondrial complex 3 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 3.

Further, for the activity of mitochondrial complex 4 of the Alzheimer's dementia model Drosophila, compared to the experimental group with which only general feed was fed, the experimental group fed with a feed supplemented with Angelica gigas Nakai extract was increased by 187.6%, and the experimental group fed with a feed supplemented with the steamed mature silkworm product powder was increased by 126.6%. On the other hand, Drosophila of the experimental group fed with a combination of the steamed mature silkworm product powder and Angelica gigas Nakai extract (Example 1) was increased by 270.4%. The result shows that a combination of the steamed mature silkworm product powder and Angelica gigas Nakai extract has a significantly higher activity increase rate, which is 1.4 to 2.1 times (82.8 to 143.8% p), which is greater than the activity increase rate of mitochondrial complex 4 of the experimental group treated with Angelica gigas Nakai extract alone or the steamed mature silkworm product powder alone. These results indicate that a combination of the steamed mature silkworm product powder and Angelica gigas Nakai extract has a synergistic effect since the steamed mature silkworm product powder and Angelica gigas Nakai extract complement each other for the activity of mitochondrial complex 4.

As described above, when the mitochondrial complex activity in the Alzheimer's dementia model Drosophila is generalized and summarized, in both the normal control and Alzheimer's dementia experimental group, the activity of the mitochondrial complex 1 to 4 of normal Drosophila fed with the steamed mature silkworm product powder alone or the Angelica gigas Nakai extract alone was significantly increased. In addition, the activity of the mitochondrial complex 1 to 4 of the experimental group treated with a combination of the steamed mature silkworm product powder and Angelica gigas Nakai extract had a significant increase (minimum 61.14% p to maximum 156.0% p) compared to those treated with the steamed mature silkworm product powder alone or the Angelica gigas Nakai extract alone, showing a synergistic effect so that it is considered to be able to significantly contribute to the prevention and treatment of Alzheimer's dementia.

### Experimental Example 7. Confirmation of the Improvement Effect of ATP Production Ability by the Treatment of the Mixture of Example 1 of the Present Invention

### 1. Experimental Method

In order to confirm the effect of the increase in mitochondrial complex activity on ATP production, the amount of ATP in the brain of Drosophila fed with only general feed and the amount of ATP in the brain of Drosophila fed with alone or in combination with the Angelica gigas Nakai extract and the steamed mature silkworm product powder were compared. It is known that ATP production decreases as aging progresses. The amounts of ATP in 7-day-old young Drosophila and 20-day-old aging Drosophila after adult emergence were compared and analyzed.

### 2. Experimental Result

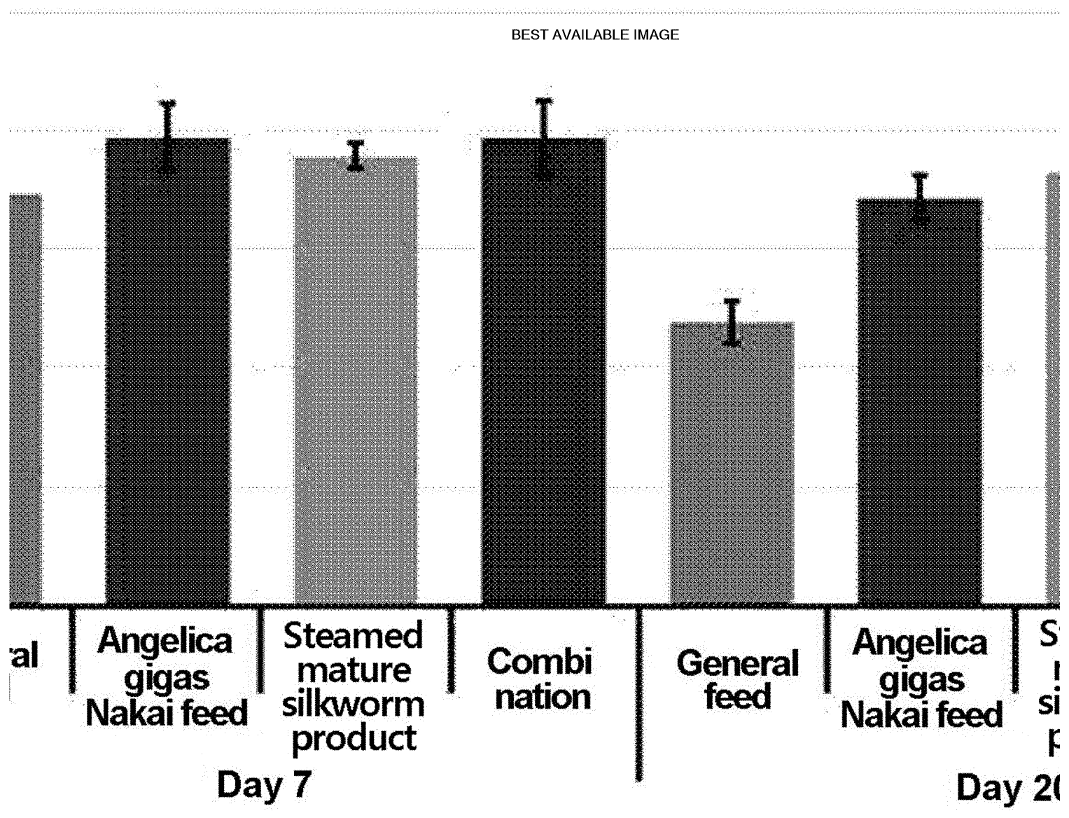

As shown in FIG. 6, the ATP amount of 20-day-old aging Drosophila in the experimental group treated with the Angelica gigas Nakai extract alone and the steamed mature silkworm product powder alone was increased to 43.9 to 52.5% compared to the Drosophila fed with only general feed. It was confirmed that although aging progressed, it showed similar vitality to the 7-day-old young Drosophila. In addition to these results, the experimental group treated with a combination of the steamed mature silkworm product powder and the Angelica gigas Nakai extract (Example 1) was also increased to 59.9% (1.1 to 1.4 times). Therefore, it was confirmed that it exhibited a significant synergistic effect showing higher vitality than the 7-day-old young Drosophila.

In detail, the 20-day-old Drosophila fed with only general feed had undergone aging, resulting in a 31.0% reduction in ATP production compared to the 7-day-old Drosophila. However, the ATP production of Drosophila fed with a feed supplemented with the Angelica gigas Nakai extract was increased by 43.9% compared to that of Drosophila fed with only general feed. Also, the ATP production of Drosophila fed with a feed supplemented with the steamed mature silkworm product powder was increased by 52.5% compared to that of Drosophila fed with only general feed. Meanwhile, the ATP production of Drosophila fed with a combination of the steamed mature silkworm product powder and the Angelica gigas Nakai extract (Example 1) was increased by 59.9%. Therefore, a combination of the steamed mature silkworm product powder and the Angelica gigas Nakai extract (Example 1) was significantly increased 1.1 to 1.4 times (7.4 to 16.0% p) higher than the increase rate of ATP production of the group treated with the Angelica gigas Nakai extract alone or the steamed mature silkworm product powder alone. This result shows that the treatment with a combination of the steamed mature silkworm product powder and the Angelica gigas Nakai extract (Example 1) had a synergistic effect of preventing a decrease in ATP production as aging progresses compared to those fed with the Angelica gigas Nakai extract alone or the steamed mature silkworm product powder alone.

### Experimental Example 8. Confirmation of the Memory Enhancement Effect on Short-Term Memory Loss by Taking the Combination of Example 1 of the Present Invention

### 1. Experimental Method

In order to confirm the memory enhancement effect on the short-term memory loss, which is a characteristic of early Alzheimer's dementia, while the experiment was performed by feeding with the mixture of Example 1, the experimental mice were treated with scopolamine, a short-term memory loss agent. Male mice aged 4 weeks after birth were administered with the steamed silkworm processed product of yellow silkworm variety of the mixture at a ratio of 1.0 g/kg body weight for 3 weeks. After 3 weeks of administration was completed, the mice were transported to the laboratory 30 minutes before the experiment on the first day of the experiment to undergo an adaptation process, and then they were acclimated for 2 minutes in a bright room of the passive avoidance test device. On the second day of the experiment, 30 minutes before the start of the experiment, mice were injected with scopolamine dissolved in saline at a weight ratio of 0.75 mg/kg. The same amount of saline was injected into the control group. After placing the mouse in the bright room of the passive avoidance device, the passage to the dark room was opened after 30 seconds, and when the tail of the mice was entered, an electric shock of 0.4 mA was applied for 4 seconds. After that, the length of time of entering the dark room was measured. On the third day of the experiment, the mice were moved to a bright room, the passage to the dark room was opened, and the length of time the tail enters was measured. At the end of the experiment, statistical analysis was performed by ANOVA and T-test on the difference between the control group and the experimental group.

### 2. Experimental Result

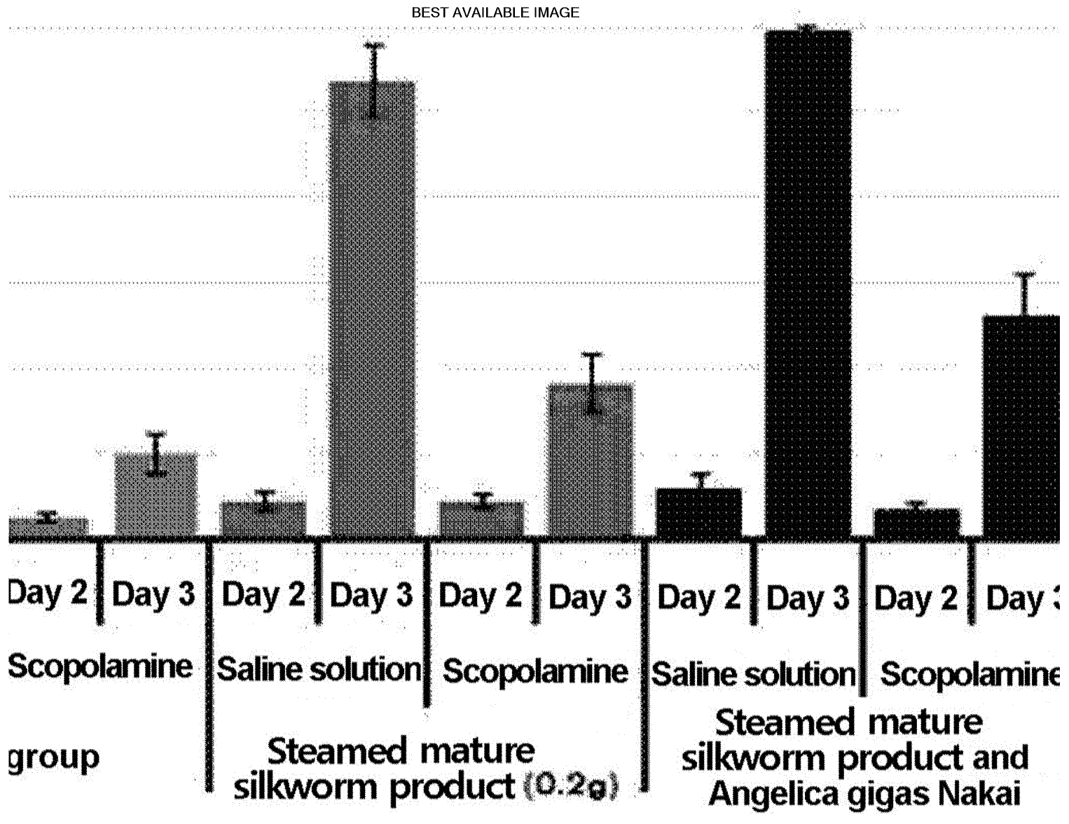

As shown in FIG. 7, in the case of the control mice fed with only general feed, both saline administration group and saline and scopolamine administration group took 20.2±5.13 seconds and 13.1±2.06 seconds, respectively to enter the dark room on the second day, so there was no significant difference. However, on the third day, 24 hours after receiving the electric shock, the saline-treated mice took 284.6±11.69 seconds to enter the dark room, indicating that they had a memory of electric shock.

In comparison, mice administered with scopolamine moved to a dark room in 50.1±11.9 seconds, showing that memory for electric shock was lost.

In comparison, in the case of mice fed with the steamed mature silkworm product powder, the staying time in the illuminated room of the saline-only administered group was 22.1±5.88 seconds on the second day and 267.6±20.5 seconds on the third day, so there was no difference compared to the control fed with only general feed.

On the other hand, the scopolamine administered experimental group had 22.2±4.1 seconds on the second day, so there was no difference. However, on the third day in which the electric shock was applied, they had 90.7±17.4 seconds, which is an 81.3% increase in memory compared to the control group fed with only general feed.

In the case of the experimental group fed with Angelica gigas Nakai extracts, the experimental group administered with the only saline had 18.8±4.2 seconds of staying in the illuminated room on the second day, and 218.2±33.4 seconds on the third day in which the electric shock was applied. There was no significant difference from the control group fed with only general feed and the group treated with the steamed mature silkworm product.

However, in the case of the short-term memory loss induction group fed with scopolamine, memory was increased by 80.6% from 17.0±4.8 seconds on the second day to 90.3±27.7 seconds on the third day in which the electric shock was applied, compared to the control group fed with only general food.

In contrast, in the case of the experimental group administered with a combination of the steamed mature silkworm product powder and the Angelica gigas Nakai extract (Example 1), the experimental group treated with the only saline had 30.3±8.7 seconds of staying in the illuminated room on the second day, so there was no statistical difference from that of the control group or other experimental groups. However, after receiving an electric shock, the time to stay in the illuminated room without entering the dark room was increased to 297.5±2.5 seconds.

In the case of the experimental group in which short-term memory loss was induced by feeding with scopolamine, they had 7.4±3.3 seconds on the second day of staying in the illuminated room, which was not different or faster than the control group or other experimental groups, but 130.1±25.4 seconds on the third day in which the electric shock was applied, so memory was increased by 159.7%.

This is because a combination of the steamed mature silkworm product powder and the Angelica gigas Nakai extract (Example 1) showed a significant memory recovery effect that was 2.0 times (76.6 to 79.1% p) higher than the short-term memory increase rate of the experimental group treated with Angelica gigas Nakai alone or the steamed mature silkworm product powder alone. This result shows that in a combination of the steamed mature silkworm product powder and the Angelica gigas Nakai extract (Example 1), the steamed mature silkworm product powder and Angelica gigas Nakai extract complement each other to improve short-term memory.

### Experimental Example 9. Confirmation of the Enhancement Effect of Spatial Memory According to the Administration of the Combination of Example 1 of the Present Invention

1) Comparison of the Enhancement Effect of Spatial Memory in Y-Maze Test According to the Administration of Combination of the Steamed Mature Silkworm Product and the Angelica gigas Nakai Extract of Example 1 of the Present Invention

### 1. Experimental Method

As for the experimental animals, an 8-week-old C57BL/6N male mouse was purchased from an experimental animal supplier (Orient, Ga-Pyung, Korea), and an environmental adaptation period of 7 days was given. All experimental animals were divided into 4 animals per cage to maintain a constant temperature (20 2° C.) and relative humidity (50 to 55%), and were reared in a laboratory environment that alternates day and night at 12 hour intervals, and a sufficient amount of drinking water and feed was supplied.

Group separation and sample treatment are as follows:

1) control group (0.25% methyl cellulose);

2) scopolamine, a short-term memory loss agent, administration group (scopolamine-injection+saline administration group);

3) 205 mg/kg of steamed mature silkworm product alone administration group (scopolamine-injection+205 mg/kg b.w. steamed mature silkworm product administration group);

4) 165 mg/kg of Angelica gigas Nakai alone administration group (scopolamine-injection+165 mg/kg b.w. steamed mature silkworm product administration group);

5) 205 and 164 mg/kg of steamed mature silkworm product and Angelica gigas Nakai administration group (scopolamine-injection+205+164 mg/kg b.w.);

6) Red ginseng administration group (308 mg/kg scopolamine-injection+Red ginseng); and/or

7) Donepezil, Alzheimer's type dementia treatment agent, administration group (scopolamine injection+1 mg/kg b.w. donepezil administration group).

The groups were pre-treated (oral-administrated) with the steamed mature silkworm product, Angelica gigas Nakai, a combination of the steamed mature silkworm product and Angelica gigas Nakai, red ginseng, and donepezil at the concentration of 205, 165, 205+164, 308, 2 mg/kg b.w., respectively for 24 days at 24 hours interval. 30 minutes before starting the behavioral experiment, scopolamine was administered intraperitoneally (1 mg/kg b.w.). 30 minutes after administration of scopolamine, the Y-maze test and behavioral evaluation were performed. In this way, behavioral evaluation was performed for a total of 7 days.

The Y-maze used in the Y-maze test consists of three arms, and each arm is 42 cm long, 3 cm wide, and 12 cm high, and the angle of contact with the three arms is 120 degrees. All experimental devices are made of black polyvinyl plastic. After each arm was designated as A, B, C, the mouse was placed on one arm and allowed to move freely for 8 minutes, and then the arm into which the mouse entered was recorded. At this time, for only the case when the tail was completely re-entered, the case of re-entering the arm that had been entered was recorded. When entering three different arms one after the other 1 point (actual alternation) was given. Alternation behavior is defined as entering into all three arms in turn and calculated by the following equation:

Alternation behavior (%)=Actual alternation/Maximum alternation×100 (maximum alternation: total number of entries−2)

### 2. Experimental Result

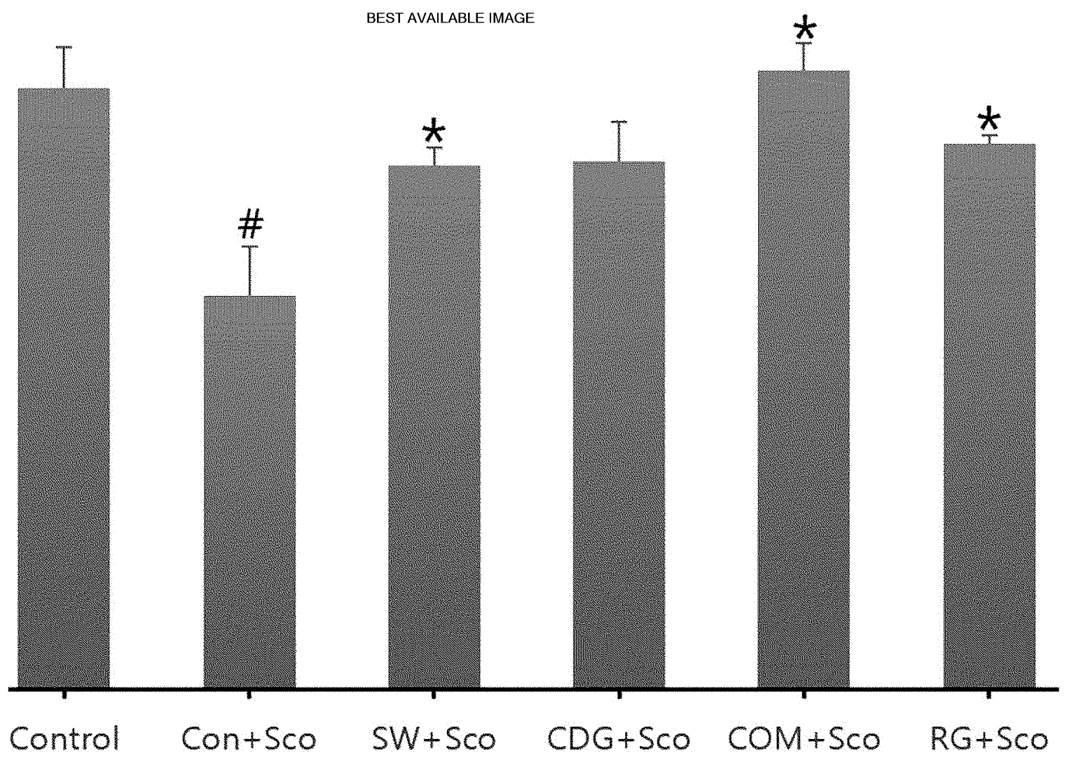

As a result of the experiment, as shown in FIG. 8, spontaneous alternation behavior by administration of scopolamine (1 mg/kg, i.p.) was statistically significantly reduced compared to the control group (p<0.05). On the other hand, the spontaneous alteration behavior by the administration with the steamed mature silkworm product and Angelica gigas Nakai extract was increased compared to the scopolamine administration group. In addition, the spontaneous alternation behavior of the group administered with the steamed mature silkworm product and combination was significantly increased significantly. Increasing these spontaneous altering behaviors indicates that learning and memory have been recovered.

2) Comparison of the Enhancement Effect of Spatial Memory in Morris Water Maze Test According to the Administration of Combination of the Steamed Mature Silkworm Product and the Angelica gigas Nakai Extract of Example 1 of the Present Invention

### 1. Experimental Method

In the art, experiments on spatial learning and cognitive improvement through the Morris water maze test are already known as a method of screening for drugs for improving dementia. This maze is 90 cm in diameter and 45 cm in height, and the diameter of the white platform is 6 cm. Around the water maze, spatial cues such as a computer system connected to a video camera and a water temperature control device were kept constant. As an experimental method, the maze was filled with water so that the height of the water was 30 cm, and the platform was installed 1 cm below the height of the water so that the mouse could not see the platform. The maze was divided into four divisions using four markers, and the maze was divided into northeast (NE), northwest (NW), southeast (SE), and southwest (SW), and a platform was installed in one quadrant of the maze.

The Morris water maze test was conducted for 6 days. On the first day, each mouse was allowed to swim freely in the maze for 1 minute so that they could adapt to the water. At this time, the platform was not installed. From the second day to the fifth day, each mouse was allowed to swim in the maze at 10 minute intervals for 1 minute, 4 times a day. The experiment method once for 4 days from the second day to the fifth day is that mice that have been on the platform already installed in the maze for 10 seconds within 1 minute finish the experiment, but mice that cannot find the platform within 1 minute or climbs on the platform for 10 seconds have been artificially placed on the platform for 10 seconds after the experiment. At this time, the position of the platform was fixed in the same place. On the sixth day, after removing the platform from the maze, the time the mouse stayed at the location where the platform was located was measured. Scopolamine was used as a forgetfulness-inducing substance and was administered intraperitoneally at 1 mg/kg 30 minutes before the first entry of water every day. All experimental data were measured and recorded using the Smart3.0 program (Noldus, Netherlands).

### 2. Experimental Result

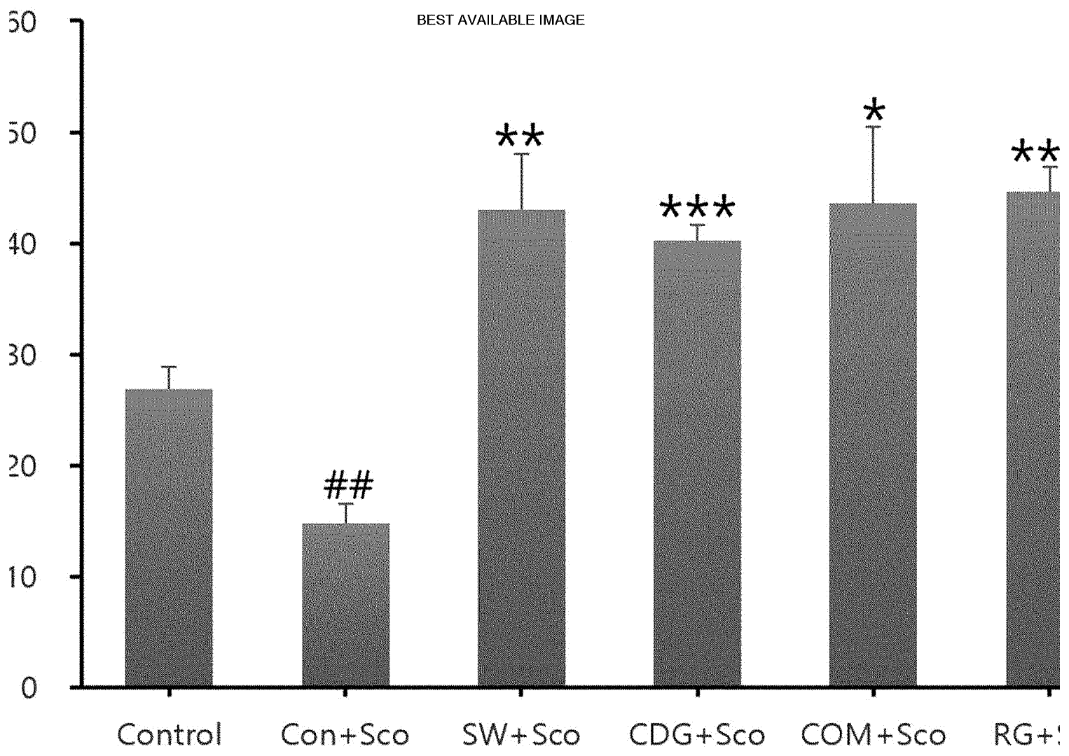

As a result of the Morris water maze test, the platform was removed on the sixth day, mice were released in the maze for 1 minute, and the time of staying in the target zone where the platform was located was measured as a ratio. As shown in FIG. 9, the control group (Control) was 26.87%; the control group+scopolamine group (Con+Sco) was 14.81%; the group administrated with Scopolamine+steamed mature silkworm product (SW+Sco) was 43.03%; the group administrated with Scopolamine+Angelica gigas Nakai (CDG+Sco) was 40.28%; the group administrated with Scopolamine+combination with steamed mature silkworm product and Angelica gigas Nakai (COM+Sco) was 43.66%; and the group administrated with Scopolamine+red ginseng memory improvement product (RG+Sco) was 44.67%. This result indicates that the group administrated with combination with the steamed mature silkworm product and Angelica gigas Nakai (COM) of the present invention had significant effects.

### Experimental Example 10. Comparison of the Improvement Effect in the Dementia Mouse Model (3×TG-AD) According to the Administration of the Combination of Example 1 of the Present Invention

1) Comparison of the Improvement Effect of Posture Control Ability in 3×TG Dementia Mouse Model According to the Administration of the Combination of the Steamed Mature Silkworm Product and Angelica gigas Nakai Extract of Example 1 of the Present Invention

### 1. Experimental Method

The 3×TG dementia mouse model is a transgenic mouse model that expresses the APP-Swedish mutation and the PSEN1 M146V mutation, which are familial Alzheimer's dementia-related mutations and the MAPT P301L mutations, which are the frontal dementia-related mutations.

In order to compare the effect on the loss of postural control ability, one of the abnormal behaviors of Alzheimer's dementia, using a combination of steamed mature silkworm product and Angelica gigas Nakai extract, which showed excellent effects in the previous experimental examples, spatial memory was tested after 3×TG dementia mice, a dementia-induced transgenic mouse model, were fed with feed including steamed mature silkworm product and Angelica gigas Nakai extract. Dementia mice were ingested at a ratio of 0.2 g/Kg body weight for steamed mature silkworm product and 0.16 g/Kg body weight for Angelica gigas Nakai extract, and then posture control ability test (mouse clasping test) was performed on mice 24 weeks old after birth.

In the mouse clasping test, a video was recorded for 10 seconds after holding the tail of the subject mouse, and then two researchers observed each video and gave a score from 0 to 4 depending on how many legs can be stretched. If all four legs cannot be stretched, 4 points are given, and if all four legs can be extended, 0 points. The results of the two analyzes were averaged. The better the posture control ability, the lower the score, close to 0.

### 2. Experimental Result

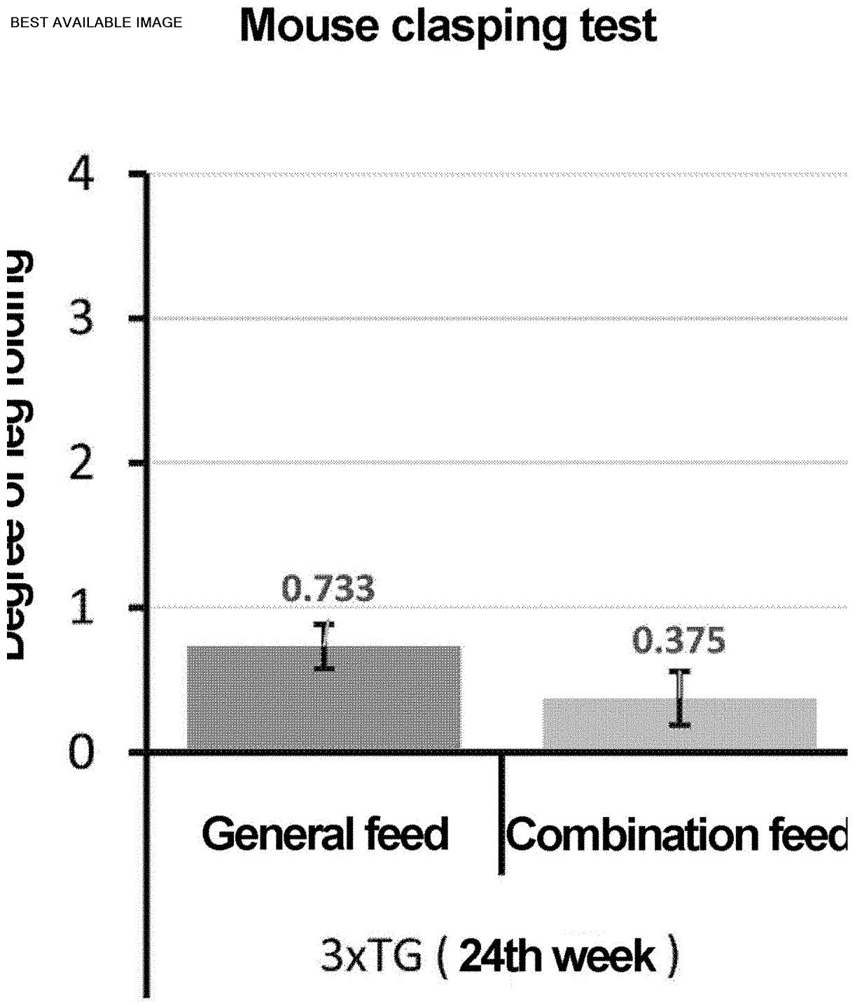

As a result of the above experiment, as shown in FIG. 10, the posture control ability of 3×TG dementia mice fed with general feed was 0.733±0.1532, and 70% or more of 3×TG mice could not properly stretch one leg. However, in 24 weeks old 3×TG dementia mice fed with a combination diet of steamed mature silkworm product and Angelica gigas Nakai extract, their postural control ability was 0.375±0.1829, which shows 48.8% improvement.

2) Comparison of the Enhancement Effect of the Recognition Ability for New Objects in 3×TG Dementia Mouse Model According to the Administration of the Combination of the Steamed Mature Silkworm Product and Angelica gigas Nakai Extract of Example 1 of the Present Invention

### 1. Experimental Method

In order to confirm whether the combination of the steamed mature silkworm product and Angelica gigas Nakai extract showing an excellent effect in the previous experimental examples can suppress the deterioration of recognition ability for new objects, one of the abnormal behaviors of 3×TG dementia mice, after dementia mice were fed with combination feed of the steamed mature silkworm product and Angelica gigas Nakai extract, novel objective recognition assay was conducted.

On the first and second days, the mice can be moved freely for 10 minutes in an empty space of 50 cm wide, 50 cm long and 50 cm high. On the third day, two identical objects were put in, and then the movement of the mice was recorded for 10 minutes. Then, the time of staying at each object was measured. On the last day, each of the old and new objects was put in, and then it was recorded for 10 minutes. Then, the time of staying at the old and new objects, respectively, was measured.

### 2. Experimental Result

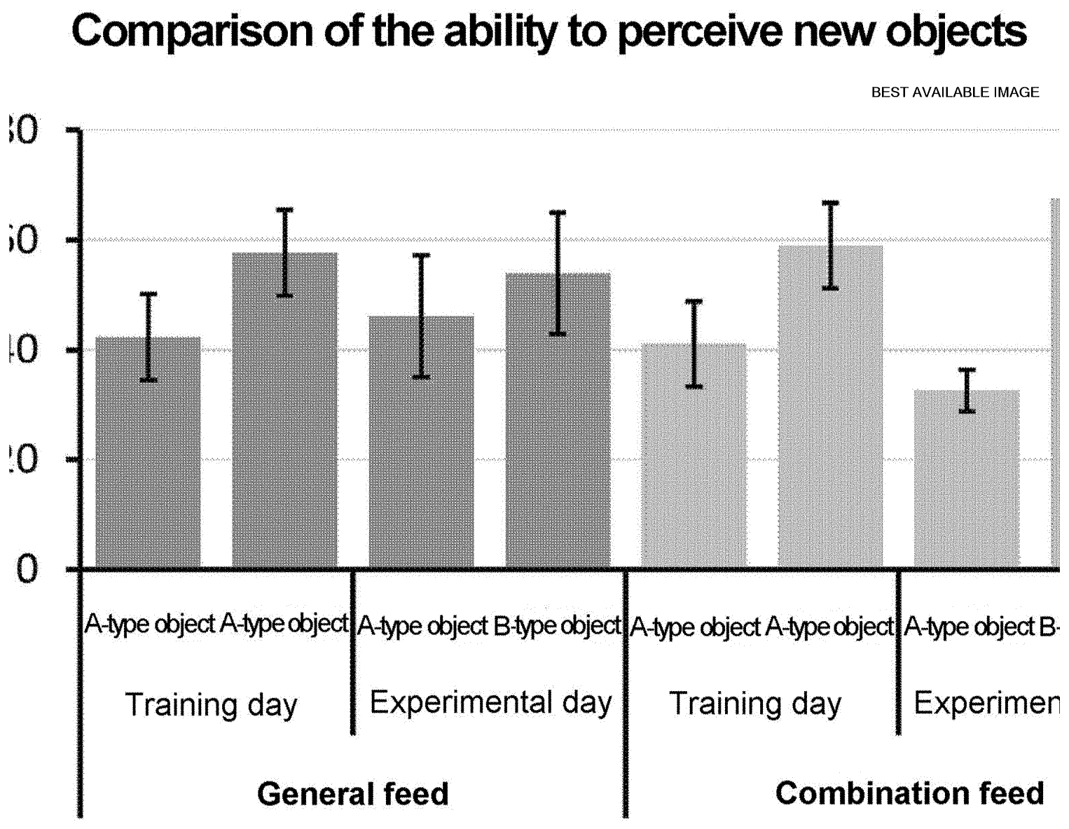

The result of the above experiment shows that as shown in FIG. 11, 3×TG dementia mice fed with a combination of steamed mature silkworm product and Angelica gigas Nakai extract had superior recognition ability for new objects compared to 3×TG dementia mice fed with general feed.

In the case of 22 weeks old 3×TG dementia mice fed with general feed, the time of staying near two identical objects on training day was 42.30±7.843% and 57.69±7.843%, respectively, so there was no statistical difference (p>0.05). When one object was changed into a new object on an experimental day, the time of staying on a new object was 53.89±11.089%, but the time of staying on an old object was 46.10±11.089%. Thus, there was no statistical difference. Meanwhile, in the dementia mice fed with a combination of steamed mature silkworm product and Angelica gigas Nakai extract, the time of staying near two identical objects on training day was 41.03±7.758% and 58.96±7.758%, respectively, so there was no statistical difference. On an experimental day, there was a statistically significant difference in interest in new or old objects, 67.46±3.819% and 32.54±3.810%, respectively. These results show that ingestion of a combination of steamed mature silkworm product and Angelica gigas Nakai extract can suppress the decrease in interest for new products in dementia mice.

3) Comparison of the Improvement Effect of Sociality in 3×TG Dementia Mouse Model According to the Administration of the Combination of the Steamed Mature Silkworm Product and Angelica gigas Nakai Extract of Example 1 of the Present Invention

### 1. Experimental Method

In order to compare effects on sociality loss, characteristic of Alzheimer's dementia, using the combination of the steamed mature silkworm product and Angelica gigas Nakai extract showing an excellent effect in the previous experimental examples, after 3×TG dementia mice were fed with the combination, the social interaction assay was performed.

The social interaction assay was performed using a mouse social interaction assay device (60 cm wide, 40 cm long, 40 cm high) divided into three spaces. The three spaces are divided by two transparent acrylic plates with holes through which the mouse can pass. In both side spaces, there is a cage with a diameter of 10 cm and a height of 6 cm made of a wire mesh that can accommodate a mouse. First, in a situation where there were no mice in both cages, the experimental mice were placed so that they could freely acclimate for 5 minutes. When the adaptation was completed, a mouse that had never met was put in one cage, and the other was left in an empty cage, and the mouse was placed in the center space and the behavior was recorded for 10 minutes. At the end, a new mouse was placed in an empty cage, and the behavior was recorded for 10 minutes. The recorded behavior was analyzed to measure the time when each experimental mouse stayed in the space with the empty cage, time when each experimental mouse stayed in the space with the mouse, time when each experimental mouse stayed in the space with the familiar mouse, and time when each experimental mouse stayed in the space with the new mouse, respectively.

### 2. Experimental Result

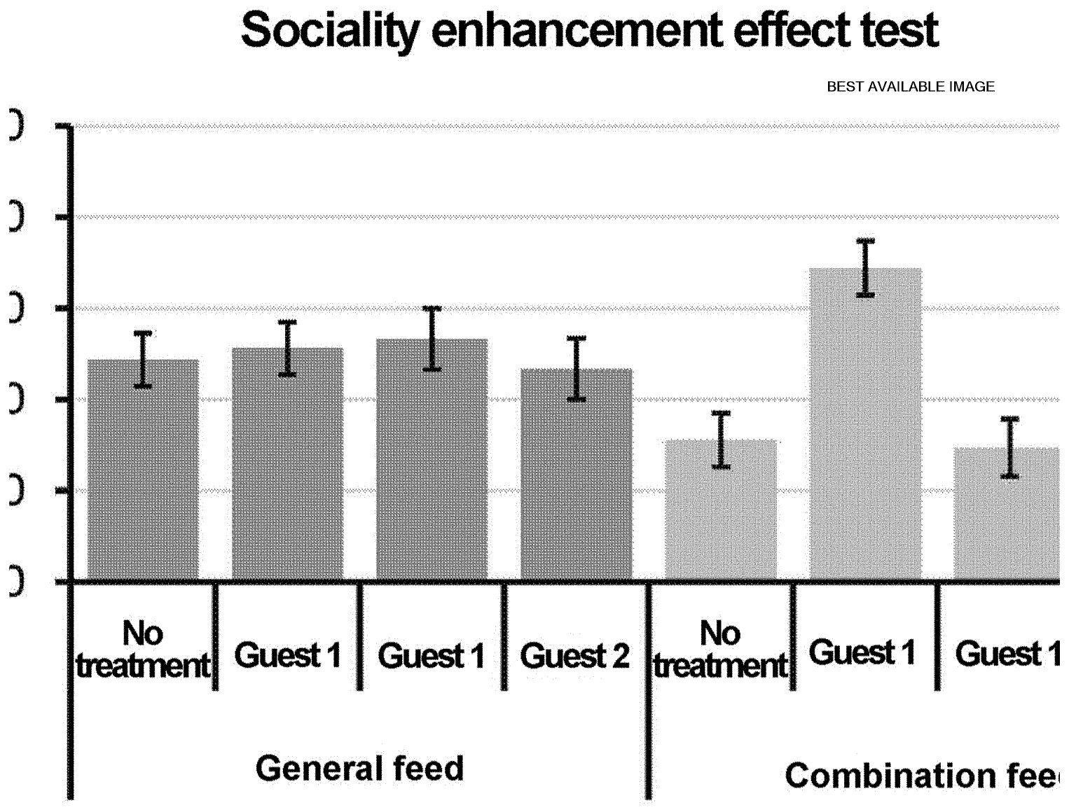

As a result of the above experiment, as shown in FIG. 12, 3×TG dementia mice fed with a combination of steamed mature silkworm product and Angelica gigas Nakai extract had superior sociality than 3×TG dementia mice fed with a general feed.

The 23 weeks old 3×TG dementia mice fed with general feed had 48.75±5.772% of their stay in the untreated space and 51.24±5.772% of their stay in the space with the unfamiliar mouse, so there is no difference. In addition, the ratio of the time to stay in the space where the new mouse was placed on the next day was 46.71±6.683%, and the ratio of the time to stay in the space where the mouse that had already met once was placed was 53.28±6.683%, so there is no difference. However, in the case of 3×TG dementia mice fed with a combination of the steamed mature silkworm product and Angelica gigas Nakai extract, the ratio of the time to stay in the space where the new mouse was placed was 68.82±5.907%. The result indicates that they stayed statistically significantly longer compared to 31.17±5.907%, which is the ratio of time to stay in the space with an empty cage. The ratio of time to stay in the space with the new mouse on the next day was 70.55±6.341%, which was statistically significantly higher compared to 29.44±6.341%, the time to stay in the space with the mouse that had already met once. These results suggest that ingestion of a combination of steamed mature silkworm product and Angelica gigas Nakai extract can improve social deficit in 3×TG dementia mice.

As described above, the present invention provides excellent memory improvement effect and prevention and treatment effect of Alzheimer's dementia when ingesting a combination of steamed mature silkworm product powder and Angelica gigas Nakai extract (Example 1), which are raw materials for food and medicine in a new form, which show a more significant synergistic effect compared to ingestion of the steamed mature silkworm product powder alone or Angelica gigas Nakai extract alone. It is expected that the steamed mature silkworm product powder and Angelica gigas Nakai extract have different mechanisms of action in the nervous system, so when they are prepared in the form of a combination formulation, they complement each other and exert a synergistic effect.

The above results indicate that it is expected to be able to fundamentally prevent and treat aging and dementia caused by aging, which is a major problem in modern society and that it is expected to help the memory improvement of various persons such as students, test-takers, and office workers. Therefore, it is expected that health functional foods and medicines for improving memory and preventing and treating dementia using the present invention are expected to be developed and marketed. Accordingly, it is expected that the effect of contributing to the development of healthy functional food production, sericulture, and Angelica production and agricultural development due to the creation of new utilization and high added value of the steamed mature silkworm product and Angelica.

### Preparation Example 1. Pharmaceutical Preparation

200 g of the mixture of Example 1 was mixed with 175.9 g of lactose, 180 g of potato starch, and 32 g of colloidal silicic acid. After adding a 10% gelatin solution to this mixture, it was pulverized and passed through a 14 mesh sieve. This was dried, and 160 g of potato starch, 50 g of talc, and 5 g of magnesium stearate were added thereto, and the resulting mixture was prepared in a form of tablet.

### Preparation Example 2. Food Preparation

### Preparation Example 2-1. Cooking Seasoning Preparation

1% by weight of the mixture of Example 1 was added to the cooking seasoning to prepare a cooking seasoning for health promotion.

### Preparation Example 2-2. Dairy Product Preparation

0.1% by weight of the mixture of Example 1 was added to milk, and various dairy products such as butter and ice cream were prepared using the milk.

### Preparation Example 2-3. Vegetable Juice Preparation

The mixture of Example 1 was added to tomato juice or carrot juice to prepare vegetable juice for health promotion.

The present invention has been described with reference to preferred embodiments, and those of ordinary skill in the technical field to which the present invention pertains can implement embodiments in different forms from the detailed description of the present invention within the essential technical scope of the present invention. Here, the essential technical scope of the present invention is described in the claims, and all differences within the scope equivalent thereto should be construed as being included in the present invention.

1. A pharmaceutical composition for improving memory, the composition comprising a steamed silkworm processed product and an Angelica extract as an active ingredient.

2. The pharmaceutical composition for improving memory of claim 1,
wherein the steamed silkworm processed product is made by heating a matured silkworm for 100 minutes to 150 minutes with steam heat at 80° C. to 125° C.

3. The pharmaceutical composition for improving memory of claim 1, wherein the steamed silkworm processed product and the Angelica extract are included in a weight ratio of 1:0.1 to 1.

4. The pharmaceutical composition for improving memory of claim 1, wherein the variety of the silkworm is Golden silk.

5. A pharmaceutical composition for preventing or treating Alzheimer's dementia, the composition comprising a steamed silkworm processed product and an Angelica extract as an active ingredient.

6. The pharmaceutical composition for preventing or treating Alzheimer's dementia of claim 5,
wherein the steamed silkworm processed product is made by heating a matured silkworm for 100 minutes to 150 minutes with steam heat at 80° C. to 125° C.

7. The pharmaceutical composition for preventing or treating Alzheimer's dementia of claim 5, wherein the steamed silkworm processed product and the Angelica extract are included in a weight ratio of 1:0.1 to 1.

8. The pharmaceutical composition for preventing or treating Alzheimer's dementia of claim 5, the variety of the silkworm is Golden silk.

9. A food composition for improving memory, the composition comprising a steamed silkworm processed product and an Angelica extract as an active ingredient.

10. The food composition for improving memory of claim 9,
wherein the steamed silkworm processed product is made by heating a matured silkworm for 100 minutes to 150 minutes with steam heat at 80° C. to 125° C.

11. The food composition for improving memory of claim 9, wherein the steamed silkworm processed product and the Angelica extract are included in a weight ratio of 1:0.1 to 1.

12. The food composition for improving memory of claim 9, wherein the variety of the silkworm is Golden silk.

13. A food composition for preventing or treating Alzheimer's dementia, the composition comprising a steamed silkworm processed product and an Angelica extract as an active ingredient.

14. The food composition for preventing or treating Alzheimer's dementia of claim 13,
wherein the steamed silkworm processed product is made by heating a matured silkworm for 100 minutes to 150 minutes with steam heat at 80° C. to 125° C.

15. The food composition for preventing or treating Alzheimer's dementia of claim 13, wherein the steamed silkworm processed product and the Angelica extract are included in a weight ratio of 1:0.1 to 1.

16. The food composition for preventing or treating Alzheimer's dementia of claim 13, wherein the variety of the silkworm is Golden silk.

17. A method for improving memory, the method comprising administering a composition comprising a steamed silkworm processed product and an Angelica extract to a subject.

18. A method for preventing or treating Alzheimer's dementia, the method comprising administering a composition comprising a steamed silkworm processed product and an Angelica extract to a subject.

19. A method for preparing a composition for improving memory, the method comprising mixing a steamed silkworm processed product and an Angelica extract.

20. The method for preparing a composition for improving memory of claim 19, wherein the steamed silkworm processed product is prepared by heating a mature silkworm for 100 minutes to 150 minutes with steam heat at 80° C. to 125° C.; and
wherein the Angelica extract is obtained by treating an Angelica with water, methanol, ethanol, or the combination thereof.

21. A method for preparing a composition for preventing or treating Alzheimer's dementia, the method comprising mixing a steamed silkworm processed product and an Angelica extract.

22. The method for preparing a composition for preventing or treating Alzheimer's dementia of claim 21, wherein the steamed silkworm processed product is prepared by heating a mature silkworm for 100 minutes to 150 minutes with steam heat at 80° C. to 125° C.; and
wherein the Angelica extract is obtained by treating an Angelica with water, methanol, ethanol, or the combination thereof.
